(19) United States
(12) Patent Application Publication
(10) Pub. No.: US 2011/0033525 A1
(43) Pub. Date: Feb. 10, 2011

(54) Diterpene Glycosides as Natural Solubilizers

(76) Inventor: Zhijun Liu, Baton Rouge, LA (US)

Correspondence Address: PATENT DEPARTMENT;TAYLOR, PORTER, BROOKS & PHILLIPS, L.L.P, P.O. BOX 2471, BATON ROUGE, LA, 70821-2471, US

(21) Appl. No.: 12/937,055

(22) PCT Filed: Apr. 13, 2009
(86) PCT No.: PCT/US09/40324

§ 371 Date: Oct. 8, 2010

Related U.S. Application Data
(60) Provisional application No. 61/044,176, filed on Apr. 11, 2008.
(60) Provisional application No. 61/099,823, filed on Sep. 24, 2008.

(51) Int. Cl. A61K 9/127; A61K 47/26; A61K 31/337; A61K 31/4375; A61K 31/165; A61K 31/192; A61K 31/7048; A61K 31/12; A61K 31/355; A61K 31/05; A61K 31/122; A61K 31/343; A61K 31/436; A61K 38/13; A61K 31/366; A61K 31/352; A61K 35/60; A61K 31/496; A61K 31/415; B82Y 5/00

(52) U.S. Cl. 424/450; 514/777; 514/449; 514/283; 514/627; 514/568; 514/27; 514/679; 514/458; 514/731; 514/678; 424/94.1; 514/468; 514/29; 514/31; 514/291; 514/20.5; 514/450; 514/463; 514/452; 424/523; 514/254.07; 514/406; 977/773; 977/915

(57) ABSTRACT

Several diterpene glycosides (e.g., rubusoside, rebaudioside, steviol monoside and stevioside) were discovered to enhance the solubility of a number of pharmaceutically and medicinally important compounds, including but not limited to, paclitaxel, camptothecin, curcumin, tanshinone HA, capsaicin, cyclosporine, erythromycin, nystatin, itraconazole, and celecoxib. The use of the diterpene glycoside rubusoside increased solubility in all tested compounds. The diterpene glycosides are a naturally occurring class of water solubility-enhancing compounds that are non-toxic and that will be useful as new complexing agents or excipients in the pharmaceutical, agricultural (e.g., solubilizing pesticides), cosmetic and food industries. Aqueous solutions by using rubusoside to increase the solubility of otherwise insoluble drugs will have several new routes of administration. In addition, aqueous solutions of therapeutic compounds with rubusoside were shown to retain the known pharmacological activity of the compounds.

(In countries other than the United States:) The benefit of the 11 Apr. 2008 filing date of U.S. provisional patent application 61/044,176 and the benefit of the 24 Sep. 2008 filing date of Unites States provisional patent application 61/099,823 are claimed under applicable treaties and conventions. (In the United States:) The benefit of the 11 Apr. 2008 filing date of U.S. provisional patent application 61/044,176 and the benefit of the 24 Sep. 2008 filing date of Unites States provisional patent application 61/099,823 are claimed under 35 U.S.C. §119(e) in the United States.

### TECHNICAL FIELD

This invention pertains to new uses for diterpene glycosides as non-toxic, natural solubilizers for use in preparing aqueous solutions of various drugs, agricultural chemicals, cosmetics, and foods.

### BACKGROUND ART

### Important Compounds Insoluble in Water

Poor aqueous solubility is a common obstacle to delivering pharmaceuticals or other bioactive compounds and is a major challenge in formulating new drug products. In a study of kinetic aqueous solubility of commercial drugs, 87% were found to have solubility in water of ≧65 μg/mL and 7%≦20 μg/mL (Lipinski, C., et al., Adv. Drug Deliv. Rev. (1997) 23:3-25). The minimum acceptable aqueous solubility for a drug is about 52 μg/mL solubility based on 1 mg/kg clinical dose and average permeability (C. A. Lipinski, J Pharm Tox Meth (2000) 44:235-249). The pharmaceutical industry has been employing various approaches to increasing water-insoluble drugs for pharmaceutical drug formulations. Commonly used approaches are the uses of one or more complexing agents (e.g., cyclodextrins), cosolvents (e.g., ethanol, polyethylene glycol), surfactants (e.g., Cremophor EL, Tween 80), emulsifiers (e.g., lecithin, glycerol), and liposome, and nanosuspension techniques, alone or in combinations. Within this group, the use of complexing agents to improve solubility of water-insoluble drugs is increasing. Complexing agents improve water solubility by forming a non-covalent stoichiometric association with the pharmaceutical drug. Currently, the main complexing agents in the pharmaceutical industry are various forms of cyclodextrins (“CDs,” molecular weight around 1135 Daltons), which form inclusion complexes with water-insoluble drug. The use of cyclodextrin inclusion complexation has successfully solubilized many insoluble drugs, including an antifungal, voriconazole, and an antipsychotic, ziprasidone mesylate, which use sulfobutylether-β-cyclodextrin as the complexing agent. The most important cyclodextrins are parent α-, β-, and γ-CD as well as two modified hydroxypropyl-β-CD and sulfobutylether-β-CD. However, even the use of cyclodextrins has its disadvantages. Some of these limitations include lack of compatibility of the drug molecules with the inclusion cavity of CDs, precipitation of the formed complexes of CD-drug during dilution (e.g., in the stomach), potential toxicity and quality control of uniform CDs, and low complexation efficiency for achieving desirable solubility effect. Therefore, new complexing agents that are superior to cyclodextrins in overcoming or reducing these limitations are needed for the formulations of pharmaceutical, cosmetic, agricultural chemicals, and foods products.

Diterpenes. Taxanes are diterpenes produced by the plants of the genus Taxus (yews) such as the Pacific Yew (Taxus brevifolia) in the family of Taxaceae. Taxanes include paclitaxel and docetaxel. Paclitaxel is the anti-cancer drug under the drug name of TAXOL® and docetaxel is used under the name of TAXOTERE® (Medicinal Natural Products—A Biosynthetic Approach, 1997, John Wiley & Sons, Chichester, England; pp 186-188). Paclitaxel is a known anti-cancer diterpenoid alkaloid and is not soluble in water. The structure of paclitaxel is shown in FIG. 1H. Therapeutic solutions of paclitaxel currently contain either an oil or dehydrated alcohol or both; or paclitaxel is bound to albumin. None of these formulations are true water solutions. Other taxanes include baccatin III, 10-deacetylbaccatin III, cephalomannine, and 10-deacetylcephalomannine. These taxanes are characterized with a four-membered oxetane ring and a complex ester side-chain in their structures. All taxane compounds have poor water solubility. (U.S. Patent Application Publication no. 2007/0032438). Other medicinally important, but insoluble or poorly soluble diterpenes include retinoids (vitamin A, retinol (vitamin A1), dehydroretinol (vitamin A2), retinoic acid, 13-cis-retinoic acid and other retinol derivatives, ginkgolides, and forsakolin (a promising drug for the treatment of glaucoma, congestive heart failure, and bronchial asthma).

Quinoline alkaloids. Quinoline alkaloids are alkaloids that possess quinoline in their structures and are terpenoid indole alkaloid modifications. Camptothecins isolated from the Camptotheca acuminata trees (Family Nyssaceae) are quinoline alkaloids. Camptothecin (CPT) is a cytotoxic alkaloid and is reported to have anti-tumor properties, perhaps by inhibiting topoisomerase 1. (See, for example, U.S. Pat. No. 4,943,579). The structure of camptothecin is shown in FIG. 1F. It has poor solubility in water (The Merck Index, 1996). Semi-synthetic analogues of camptothecins such as topotecan and irinotecan are approved chemotherapeutic drugs. Natural camptothecins include camptothecin, 10-hydroxycamptothecin, methoxycamptothecin, and 9-nitrocamptothecin. None of the natural camptothecins are water soluble (see, for example, US Patent Application Publication no. 2008/0242691). Camptothecins have broad-spectrum anti-cancer activity, but poor water solubility has limited direct uses as chemotherapeutic agents. Other quinoline alkaloids include the long recognized anti-malarial drugs quinine, quinidine, cinchonidine, and cinchonine.

Phenylalanine-derived alkaloids. Phenylalanine-derived alkaloids are compounds that either possess or derive from phenylalanine ring structures, e.g., capsaicin and dihydrocapsaicin. Capsaicin (CAP) is a pungent phenylalanine alkaloid derived from chili peppers and is known to desensitize nerve receptors. The structure of capsaicin is shown in FIG. 1G. It is practically insoluble in cold water (The Merck Index, 1996).

Hydrolysable Tannins. Hydrolysable tannins include gallotannins, which include gallic acid and compounds with gallic acid as the basic unit, and ellagitannins, which include ellagic acid and compounds with ellagic acid as the basic unit. The structure of gallic acid is shown in FIG. 1A. Gallic acid is reported to be both an antioxidant and antiangiogenic agent (See, for example, Published International Application WO 2005/000330). Gallic acid is sparingly soluble (about 11 mg/ml) in water at room temperature, and the solution is light sensitive (The Merck Index, 1996).

Flavonoids. Flavonoids are polyphenolic compounds, and include flavonoids derived from a 2-phenylchromen-4-one (2-phenyl-1,4-benzopyrone) structure, isoflavonoids derived from a 3-phenylchromen-4-one (3-phenyl-1,4-benzopyrone) structure, and neoflavonoids derived from a 4-phenylcoumarine (4-phenyl-1,2-benzopyrone) structure. Many chalcones act as precursors to form a vast variety of flavonoids. The most noticeable subclasses of flavonoids include flavonones (e.g., naringenin and eriodictyol), flavones (e.g., apigenin and luteolin), dihydroflavonols (e.g., dihydrokaempferol and dihydroquercetin), flavonols (e.g., kaempferol and quercetin), flavandiols and leucoanthocyanidins (e.g., leucopelargonidin and leucocyanidin), water-soluble catechins (e.g., afzalechin and catechin), moderately soluble anthocyanidins (e.g., pelargonidin and cyaniding), as well as flavonol glycosides (e.g., rutin) and flavonone glycosides (e.g., hesperidin, neohesperidin and naringin). Isoflavonoids include, for example, the compounds daidzein and genistein (phyto-oestrogens). Neoflavonoids include, for example, the compounds of coumestrol, rotenone, and pisatin. A specific example of a flavonol glycoside is rutin, a light-yellow colored compound, which is a potent anti-oxidant that inhibits some cancers and reduces the symptoms of haemophilia. The structure of rutin is shown in FIG. 1B. Rutin has also a veterinary use in the management of chylothorax in dogs and cats. The obstacle to all these potential uses is its poor solubility in water (125 μg/ml; The Merck Index, 1966).

Curcuminoids/phenols. Curcuminoids/phenols are a class of compounds found in turmeric spice from the plant, Curcuma longa, of the ginger family. Curcuminoids include, for example, curcumin, desmethoxycurcumin, and bis-desmethoxycurcumin. Other phenols include, for example, tocopherols (vitamin E), propofol, and gingerols. Curcumin is an orange-yellow pigment that is found in the rhizome of Curcuma longa, the source of the spice turmeric. The structure of curcumin is shown in FIG. 1E. Curcumin has been reported to have several beneficial properties, including promotion of general health, anti-inflammatory and antimicrobial properties, and treatment for digestive disorders. (See, for example, U.S. Pat. No. 6,673,843) Curcumin is a lipophilic compound that is insoluble in water (The Merck Index, 1996). Alpha-tocopherol, one of the most potent forms of Vitamin E, is a lipid-soluble phenol compound that is not soluble in water. Its structure is shown in FIG. 1N. Gingerols are lipid-soluble phenol compounds primarily isolated from the root of ginger (Zingiber officinale). The structure of 6-gingerol is shown in FIG. 1P. Gingerols (e.g., 6-gingerol) may reduce nausea caused by motion sickness or pregnancy and may also relieve migraine.

Propofol is a drug for anesthetic and hypnotic uses. Currently, there are two drug forms using propofol. Its structure is shown in FIG. 1O. Propofol is formulated as an emulsion of a soya oil/propofol mixture in water. Newer generic formulations contain sodium metabisulfite or benzyl alcohol. Propofol emulsion (also known as “milk of amnesia”) is a highly opaque white fluid. The drug is sold as 200 mg propofol in 20 mL emusifier (1%). The other drug form of propofol is a water-soluble form of the drug, fospropofol.

Quinones. Quinones are a class of compounds having a fully conjugated cyclic dione structure. This class includes, for example, ubiquinones (coenzyme Q, such as coenzyme Q10), plastoquinones, anthraquinones (e.g., rhein, emodin, alizarin, and lucidin), phenanthraquinones (e.g., cryptotanshinone, tanshinone I, tanshinone IIA, and dihydrotanshinone), and di-anthraquinones (e.g., sennosides A and B). For example, tanshinone IIA is one of the natural analogues of tanshinone. The structure of tanshinone IIA is shown in FIG. 1C. Tanshinones have been reported to have various physiological activities from attenuating hypertrophy in cardiac myocytes to aiding in treatment of obesity. (See, for example, U.S. Patent Application Publication 2007/0248698). Tanshinone IIA (as well as other tanshinones such as tanshinone I) is soluble in methanol but insoluble in water.

Another quinone is coenzyme Q10 (often abbreviated as CoQ10), a benzoquinone. The structure of CoQ10 is shown in FIG. 1D. This oil-soluble vitamin-like substance is a component of an electron transport chain in aerobic cellular respiration. CoQ10 acts as an antioxidant and is often used as a dietary supplement. The problems with CoQ10 are its insolubility in water and low bioavailability. Several formulations have been developed and tested on animals or humans including attempts to reduce the particle size and increase surface area of the compound, soft-gel capsules with CoQ10 in oil suspension, the use of aqueous dispersion of solid CoQ10 with tyloxapol polymer, formulations based on various solubilising agents, i.e. hydrogenated lecithin, and complexation with cyclodextrins, carriers like liposomes, nanoparticles, and dendrimers. Solubilizing CoQ10 in a water solution could have many uses as new medical treatments, including the administration by injection.

Microlides. Microlides are a large family of compounds, many with antibiotic activity, characterized by a macrocyclic lactone ring typically 12-, 14-, or 16-membered (reflecting the number of units used), but can also be even larger polyene macrolides with microlide ring size ranging from 26 to 38-membered. Some examples of typical macrolides are erythromycins (14-membered) from Streptomyces erythreus, oleandomycin (14-membered) from Streptomyces antibioticus, spiramycin I, II, and III (16-membered) from Streptomyces ambofaciens, tylosin (16-membered) from Streptomyces fradiae, and avermectins (16-membered with a long polyketide chain). Some examples of polyene macrolides are amphotericin B from Streptomyces nodosus, nystatin from Streptomyces noursei, tacrolimus (23-membered) from Streptomyces tsukubaensis, and rapamycin (sirolimus; 31-membered).

Erythromycin is a macrolide antibiotic (polyketide). Its structure is shown in FIG. 1J. Erythromycin has an antimicrobial spectrum similar to or slightly wider than that of penicillin, and is often used for people who have an allergy to penicillins. For respiratory tract infections, it has better coverage of atypical organisms, including mycoplasma and Legionella.

Amphotericin B is a polyene antifungal, antibiotic from Streptomyses and has antimicrobial spectrum covering yeast and other fungi. It is a yellowish powder that is insoluble in water. The structure of amphotericin B is shown in FIG. 1V. Examples of applications of Amphotericin B: (1) antifungal: use of intravenous infusion of liposomal or lipid complex preparations of Amphotericin B to treat fungal disease, e.g., thrush; (2) use in tissue culture to prevent fungi from contaminating cell cultures. It is usually sold in a concentrated lipid complex/liposomal solution, either on its own or in combination with the antibiotics penicillin and streptomycin; (3) use as an antiprotozoal drug in otherwise untreatable parasitic protozoan infections such as visceral leishmaniasis and primary amoebic meningoencephalitis; and (4) use as an antibiotic in febrile, immunocompromised patients who do not respond to broad-spectrum antibiotics. An aqueous formulation of amphotericin B would offer new ways to administer this important drug, including intravenous use.

Nystatin is polyene macrolide from Streptomyces noursei which increases the permeability of the cell membrane of sensitive fungi by binding to sterols. It has an antimicrobial spectrum against yeasts and molds. It is a light yellowish powder, and is relatively insoluble in water. The structure of nystatin is shown in FIG. 1K. Current administration orally or topically relies on formulations based on lipids. Examples of applications of nystatin include cutaneous, vaginal, mucosal and esophageal Candida infections; and as prophylaxis in patients who are at risk for fungal infections. A water soluble formulation will allow new uses and routes of administration.

Rapamycin, also known as Sirolimus, is an immunosuppressant drug used to prevent rejection in organ transplantation; it is especially useful in kidney transplants. The structure is shown in FIG. 1U. Rapamycin is a macrolide originally developed as an antifungal agent, but later as a potent immunosuppressive and antiproliferative drug. Recently, rapamycin has been the subject of research and development as an inhibitor of the mammalian target of rapamycin (mTOR) for the treatment of cancer (e.g., leukemia). Rapamycin is not soluble in water. An oral solution drug containing Sirolimus formulated in phosal 50 PG and Tween 80 is currently used to prevent rejection in organ transplantation. A water solution containing therapeutic amounts of rapamycin has not been available.

Cyclic Peptides. Cyclic peptides are a class of antibiotic compounds composed of cyclic peptides produced mostly by fungi such as Cylindrocarpon lucidum and Tolypocladium inflatum. Examples of cyclic peptide compounds that are water insoluble are cyclosporins, polymyxins, tyrothricin, gramicidins, capreomycin, vancomycin, cephalosporins, and cephamycins. Cyclosporin A, also known as cyclosporine, is a fungal metabolite possessing potent immunosuppressive properties. It is a white powder that is insoluble in water. The structure of Cyclosporin A is shown in FIG. 1I. Cyclosporin A is administered orally and by injection in non-aqueous compositions, and current application relies upon suspensions and emulsions of the drug. Examples of applications of cyclosporin include an immunosuppressant drug in organ transplants to reduce the activity of the patient's immune system; use for several autoimmune disorders, inducing psoriasis, severe atopic dermatitis, and rheumatoid arthritis and related diseases; use as a neuroprotective agent in conditions such as traumatic brain injury; and use in several veterinary medicines, for example, keratoconjunctivitis sicca (“dry eye”) in dogs; perineal fistulas; atopic dermatitis in dogs; immune-mediated hemolytic anemia; discoid lupus erythemathosus (topical use); feline asthma; german shepherd pannus (ophthalmic preparation); and kidney transplantation.

Sesquiterpene lactones. Sesquiterpene lactones are a class of sesquiterpenes (15-carbon compounds) containing a lactone. Examples of insoluble sesquiterpenes are artemisinin (a new, highly-effective anti-malarial compound), dihydroartemissinin, and bilobalide (isolated from Ginkgo biloba).

Artemisinin is a sesquiterpene lactone drug used to treat multi-drug resistant strains of falciparum malaria. Artemisinin is isolated from the plant Artemisia annua, but can also be synthesized from artemisinic acid. Its structure is shown in FIG. 1L. Artemisinin is poorly soluble, which limits its bioavailability. Semi-synthetic derivatives of artemisinin, including artemether and artesunate, have been developed. However, their activity is not long-lasting, with significant decreases in effectiveness after one to two hours. To counter this drawback, artemisinin is given with lumefantrine (also known as benflumetol) to treat uncomplicated falciparum malaria. Lumefantrine has a half-life of about 3 to 6 days. Such a treatment is called ACT (artemisinin-based combination therapy); other examples are artemether-lumefantrine, artesunate-mefloquine, artesunate-amodiaquine, and artesunate-sulfadoxine/pyrimethamine. Recent trials have shown that ACT is more than 90% effective, with recovery from malaria after three days, even with chloroquine-resistant Plasmodium falciparum. A water solution of artemisinin would be highly desirable for direct parenteral applications.

Lignans. Lignans are a class of compounds in which two phenylpropane coniferyl alcohol monomer units are coupled at the central carbon of the side-chain (lignans) or at another location (neolignans). Examples of lignans are podophyllotoxin (isolated from American Mayapple), 4′-demethylpodophyllotoxin, beta-peltatin, alpha-peltatin, desoxypodophyllotoxin, podophyllotoxone, matairesinol, yatein, and pinoresinol. Podophyllotoxin, also known as codylox or podofilox, is a lignan compound, and a non-alkaloid toxin isolated from the rhizome of American Mayapple (Podophyllum peltatum). Its structure is shown in FIG. 1M. Podophyllotoxin can also be synthesized biologically from two molecules of coniferyl alcohol. Podophyllotoxin is the pharmacological precursor for the important anti-cancer drug etoposide. It is also administered to treat genital warts. Podophylotoxin is poorly soluble in water, and a water solution containing a pharmaceutically effective amount has not been available.

Flavonolignans. Flavonolignans are a class of compounds structurally combined from flavonoid and lignan. These include compounds such as silybin, isosilybin, and silychristin (seen in the plant of milk thistle (Silybum marianum) from the family of Compositae. Silybin, also known as Silibinin, is the major active constituent of silymarin, the mixture of flavonolignans extracted from milk thistle (Silybum marianum). The structure of silybin is shown in FIG. 1Q. Studies suggest that silybin has hepatoprotective (antihepatotoxic) properties and anti-cancer effects against human prostate adenocarcinoma cells, estrogen-dependent and estrogen-independent human breast carcinoma cells, human ectocervical carcinoma cells, human colon cancer cells, and both small and nonsmall human lung carcinoma cells. Poor water solubility and bioavailability of silymarin led to the development of enhanced formulations. Silipide (trade name SILIPHOS®), a complex of silymarin and phosphatidylcholine (lecithin), is about ten times more bioavailable than silymarin. It has been also reported that silymarin inclusion complex with β-cyclodextrin is much more soluble than silymarin itself. Glycosides of silybin show better water solubility and even stronger hepatoprotective effects. However, an aqueous solution of silybin in pharmaceutically acceptable amount, in its original and unmodified structure, has not been available for parenteral administrations.

Lipids. Other water insoluble therapeutic compounds or mixtures of compounds include lipids, e.g. fatty acids in fish oil. Some of the beneficial components of fish oil (i.e., omega-3 fatty acids, including eicosapentaenoic acid and docosahexaenoic acid) are shown in FIG. 1R. Fish oil has been widely used as a neuroprotectant.

Azole. An azole is a class of five-membered nitrogen heterocyclic ring compounds containing at least one other noncarbon atom, for example, a nitrogen, sulfur or oxygen (Eicher, T.; Hauptmann, S. (2nd ed. 2003). The Chemistry of Heterocycles: Structure, Reactions, Syntheses, and Applications. Wiley-VCH. ISBN 3527307206). Itraconazole is a triazole with antifungal activities. The structure of itraconazole is shown in FIG. 1S. Other triazole antifungal drugs include fluconazole, isavuconazole, voriconazole, pramiconazole, posaconazole, ravuconazole, fluconazole, fosfluconazole, epoxiconazole, triadimenol, propiconazole, metconazole, cyproconazole, tebuconazole, flusilazole and paclobutrazol. These compounds are practically insoluble in water (e.g., itraconazole, The Merck Index, 1996, p. 895). Itraconazole has relatively low bioavailability after oral administration. Some improvement has been made, for example, in SPORANOX® using cyclodextrin complexation and propylene glycol to deliver the drug via intravenous infusion. True aqueous compositions of itraconazole have been limited by the poor water solubility.

Celecoxib is a pyrazole (a rare alkaloid), a compound that targets cyclooxygenase (COX) enzymes. The structure of celecoxib is shown in FIG. 1T. In medicine, pyrazoles are used for their analgesic, anti-inflammatory, antipyretic, antiarrhythmic, tranquilizing, muscle relaxing, psychoanaleptic, anticonvulsant, monoamineoxidase inhibiting, antidiabetic and antibacterial activities. Celecoxib is a COX-2 inhibitor. Celecoxib has poor solubility in water which reduces its bioavailability. True water solutions of celecoxib have not been reported.

All of the above and many other pharmaceutically active compounds are relatively insoluble in water. The potential use of these agents in therapy could be increased if the compounds could be made soluble in an aqueous solution.

Diterpene Glycosides

Natural terpene glycosides are well known and exist in a variety of plant sources. They generally are terpene aglycons attached to at least one glucose or other simple sugars (e.g., xylose or galactose), and the most common forms are monoterpene glycosides, diterpene glucosides, and triterpene glucosides. Many of these compounds are known to be non-toxic and natural sweeteners. (U.S. Published Patent Application No. 2006/000305053; and Chinese Patent No. 1723981). Examples of diterpene glycosides include rubusoside, rebaudioside, stevioside, and steviol monoside. Rubusoside A is a diterpene glycoside mainly from Chinese sweet leaf tea leaves (Rubus suavissimus; Rosaceae). Rubusoside A has a molecular formula C₃₂H₅₀O₁₃ and molecular weight of 642.73. The structure of rubusoside is shown in FIG. 2. (From T. Tanaka et al., Rubusoside (b-D-glucosyl ester of 13-O-b-D-glucosyl-steviol), a sweet principle of Rubus chingii Hu (Rosacease), Agricultural and Biological Chemistry, vol. 45(9), pp. 2165-6, 1981). Rubusoside also has good solubility in water, alcohol and acetone ethyl acetate. The compound as shown in FIG. 2 is a diterpene aglycone with two glucose molecules attached.

Another diterpene glycoside that is isolated from the Chinese sweet leaf tea (Rubus suavissimus; Rosaceae) and from stevia leaves (Stevia rebaudiana; Asteraceae) is steviol monoside. The structure of steviol monoside has only one glucose molecule (FIG. 5) rather than two as in rubusoside (FIG. 2). Steviol monoside can be isolated from the sweet leaf tea, stevia leaves, or be obtained through the partial acid or alkaline hydrolysis of rubusoside to cleave one glucose molecule. Unlike rubusoside, steviol monoside is not a dominant diterpene glycoside in the sweet leaf tea or stevia plant.

Stevioside is a diterpene glycoside that is isolated from the Stevia leaf (Stevia rebaudiana; Asteraceae). Stevioside has a molecular formula C₃₈H₆₀O₁₈ and a molecular weight of 804. The structure is shown in FIG. 3. The compound as shown is a diterpene aglycone with three glucose molecules. In pure form, it is a crystal or white powder. Another diterpene glycoside that is isolated from the Stevia leaf is rebaudioside, which exists in several forms, including rebaudioside A, rebaudioside B, rebaudioside C, rebaudioside D, rebaudioside E, and rebaudioside F. The structure of rebaudioside A is shown in FIG. 4. The compound as shown is a diterpene aglycone with four glucose molecules. In pure form, it is a white powder.

Other diterpene that contain various numbers of glucose moieties are known in the art. These compounds include: paniculoside IV, suaviosides A, B, C₁, D₁, D₂, E, F, G, H, I, and J (FIG. 5) as identified by Ohtani et al. (1992, Phytochemistry 31(5): 1553-1559), and goshonosides F₁ to F₅ (FIG. 6) as identified by Seto et al. (1984, Phytochemistry 23 (12): 2829-2834). Although many diterpene glycosides such as stevioside, rebaudioside A, rubusoside, steviol monoside, and suavioside B, G, I, J, and H taste sweet, other diterpene glycosides are tasteless or bitter. For examples, paniculoside IV is tasteless, suavioside C₁ tastes bitter, suavioside D₁ is tasteless, suavioside D₂ tastes bitter, suavioside E is tasteless, and suavioside F tastes bitter as indicated by Ohtani et al. (1992, Phytochemistry 31(5): 1553-1559).

U.S. Published Patent Application No. 2002/0076426 discloses terpene alcohol ethoxylates as solubilizers in pharmaceutical and food preparations.

Chinese Patent No. 1723981 discloses that an extract containing triterpene glycosides (mogrosides) isolated from Momordica grosvenoiri fruit was used to replace sucrose or other sweeteners in manufacturing pills, granules, tablets, capsules or solutions of traditional Chinese medicine.

### DISCLOSURE OF INVENTION

I have discovered that several natural diterpene glycosides (including, e.g., rubusoside, rebaudioside A, stevioside, and steviol monoside) enhanced the solubility of a number of pharmaceutically and medicinally important compounds of several structural classes, including but not limited to, the important water-insoluble drugs of paclitaxel, camptothecin, curcumin, tanshinone HA, capsaicin, cyclosporine, erythromycin, nystatin, itraconazole, and celecoxib. The use of the diterpene glycoside rubusoside increased solubility of all tested compounds from about 5-fold to over 1000-fold, depending on the compound. In addition, photostability of at least one compound was enhanced. The rubusoside-paxlitaxel water solution and a rubusoside-camptothecin water solution were shown to retain cytotoxic activity against cancer cells. In addition, a rubusoside-curcumin water solution was shown to retain its antibiotic activity. The diterpene glycosides are a naturally occurring class of water solubility-enhancing compounds that are non-toxic and that will be useful in the pharmaceutical, agricultural, cosmetic, and food industries.

### BRIEF DESCRIPTION OF DRAWINGS

FIGS. 1A to 1V illustrates the structures of representative compounds of several classes of compounds that are known to have low water solubility, and that have been shown to be solubilized using a diterpene glycoside, including gallic acid (FIG. 1A), rutin (FIG. 1B), tanshinone HA (FIG. 1C), Co-Q10 (FIG. 1D), curcumin (FIG. 1E), camptothecin (FIG. 1F), capsaicin (FIG. 1G), paclitaxel (FIG. 1H), cyclosporin A (FIG. 1I), erythromycin (FIG. 1J), nystatin (FIG. 1K), artemisinin (FIG. 1L), podophyllotoxin (FIG. 1M), alpha-tocopherol (FIG. 1N), propofol (FIG. 1O), 6-gingerol (FIG. 1P), silybin (FIG. 1Q), omega-3 fatty acids (eicosapentaenoic acid and docosahexaenoic acid) (FIG. 1R), itraconazole (FIG. 1S), celecoxib (FIG. 1T), rapamycin (FIG. 1U), and amphotericin B (FIG. 1V).

FIG. 2 illustrates the structure of rubusoside, a diterpene glycoside isolated from Chinese sweet leaf tea.

FIG. 3 illustrates the structure of stevioside, a diterpene glycoside isolated from the Stevia leaf.

FIG. 4 illustrates the structure of rebaudioside A, another diterpene glycoside isolated from Stevia leaf.

FIG. 5 illustrates the structures of several diterpene glycosides isolated from Rubus or Stevia plants.

FIG. 6 illustrates the structures of several diterpene glucosides isolated from Rubus or Stevia plants.

FIG. 7 illustrates the results of high performance liquid chromatography indicating tanshione HA dissolved in 10% rubusoside water solution (upper), 20% rubusoside water solution (middle), and in methanol (lower).

FIG. 8 illustrates the results of cellular proliferation assays using human pancreatic cancer cells (PANC-1) to test the inhibitory activity of the aqueous solutions of paclitaxel (TXL) and campthotecin (CPT), each solubilized with rubusoside.

FIG. 9 illustrates the results of cellular proliferation assays using human lung (A549) and prostate (PC3) cancer cells to test the inhibitory activity of aqueous solutions of paclitaxel (TXL100) and camptothecin (CPT70), each solubilized with rubusoside.

FIG. 10 illustrates results of high performance liquid chromatography analysis on various solutions of curcumin dissolved in several solubilizers (5% rebaudioside, 5% stevioside, 5% rubusoside (both water and PBS), 100% methanol, and 5% ethanol.

FIG. 11 illustrates results of high performance liquid chromatography analysis on various solutions prepared with curcumin dissolved in various solvents (10% rubusoside (RUB10), 10% beta-cyclodextrin (BCD10), 10% polyethylene glycol (PEG10), 10% ethanol (ETOH10), and 10% dimethyl sulfoxide (DMSO10)).

FIG. 12 illustrates results of high performance liquid chromatography analysis on solutions of curcumin (CUR) and an extract of sweet leaf tea at two concentrations (1% and 5%), the extract containing rubusoside (RUB) and steviol monoside (SM).

FIG. 13 illustrates results of high performance liquid chromatography analysis on a solution containing curcumin (5) (also demethyoxycurcumin (4)) and 5% w/v of a mixture containing various steviol glycosides solubilizers (rebaudioside A (1), stevioside (2), rubusoside (3)).

FIG. 14 illustrates results of high performance liquid chromatography analysis on a solution containing curcumin and 10% w/v rubusoside (RUB10) and a solution containing curcumin and a mixture of 10% rubusoside and rebaudioside A (1:1 w/w) (CUR-SFA5C5).

FIG. 15 illustrates results of high performance liquid chromatography analysis on coenzyme Q10 (CoQ10) dissolved in an anhydrous ethanol solution (upper chromatogram) and an aqueous solution of 10% w/v rubusoside (lower chromatogram).

FIG. 16 illustrates results of high performance liquid chromatography analysis on three propofol solutions: one with water and 10% rubusoside; one with water alone; and one in methanol.

FIG. 17 illustrates results of high performance liquid chromatography analysis of two fish oil solutions: one with water alone (FO-SFAO) and one with water and 10% rubusoside (w/v) (FO-SFA10).

FIG. 18 illustrates results of high performance liquid chromatography analysis of three itraconazole solutions: methanol solution of 180 μg/ml itraconazole (ICZ Reference), itraconazole in water alone (ICZ−Solubilizer), and one with water and 10% rubusoside (ICZ+Solubilizer).

FIG. 19 illustrates results of high performance liquid chromatography analysis of three celecoxib solutions: methanol solution of 420 μg/ml celecoxib (CEL in Methanol), celecoxib in water alone (CEL in Water), and one with water and 10% rubusoside (CEL+10% Solubilizer).

### MODES FOR CARRYING OUT THE INVENTION

Several important organic compounds are insoluble in water or have very low solubility. I have tested many of these therapeutic compounds from several classes of chemical structures and found that natural solubilizers based on diterpene glycosides have increased the aqueous solubility of all compounds tested. I have found a method for enhancing the solubility of an organic compound which is insoluble or sparingly soluble in water, said method comprising mixing said compound with water and with a diterpene glycoside in a concentration sufficient to increase the solubility of the compound in water by a factor of 2 or more. The solubility for the organic compounds in some cases has been increased by a factor of 5 or more, in others by a factor of 10 or more, in others by a factor of 20 or more, in others by a factor of 50 or more, in others by a factor of 100 or more, and in others by a factor of 1000 or more.

In addition, a new composition has been discovered comprising an aqueous solution of an organic compound having low solubility in water, and a diterpene glycoside; wherein the concentration of said diterpene glycoside is sufficient to increase the solubility of said compound in water by a factor of 2 or more above what the solubility of said compound would be in an otherwise identical composition lacking said diterpene glycoside. The solubility for the organic compounds in some cases has been increased by a factor of 5 or more, in others by a factor of 10 or more, in others by a factor of 20 or more, in others by a factor of 50 or more, in others by a factor of 100 or more, and in others by a factor of 1000 or more. The solubilizers can be used in concentrations from 1% to 100% w/v. The solubilzer solutions were found to be particularly effective from about 5 to about 40% w/v solubilizer. The concentration of the solubilizer will determine the amount of the drug that will be dissolved. Thus the concentration will depend on the desired dose of the drug to be administered.

I have discovered diterpene glycosides as new solubilizing agents for creating new pharmaceutical, cosmetic, agricultural and food formulations instead of the commonly used cyclodextrins. Without being bound by this theory, it is believed that the improved solubility of water-insoluble drugs is a result of the formation of diterpene glycoside (dTGs)-drug complexes, which are water soluble. The driving forces for the formation of the dTG-drug complexes may include London dispersion forces (an induced dipole-induced dipole attraction), dipolar forces (including hydrogen-bonding), ionic (electrostatic) forces, and/or hydrophobic effects as described in R. Liu, Water-insoluble drug formulation, Second Edition, pp 133-160, 2008, CRC Press, Boca Raton, Fla. Depending on the drug molecule, solubilization power of the dTGs will vary depending on the driving force in forming each intermolecular complexation.

Without being bound by this theory, it is believed that the formation of the dTG-drug complexes in aqueous solutions may be driven by similar forces proposed for cyclodextrins in the formation of inclusion complexes. In addition to the driving forces above, van der Waals forces (the attractive or repulsive force between molecules or between parts of the same molecule) may be involved. The difference between the CD-drug inclusion complexes and the dTG-drug complexes may be attributable to their geometrical structures. Rather than forming a circle with a hydrophobic cavity similar to the CDs, the dTGs may form a uniform network, with the hydrophilic glucose molecules connecting to each other to form a backbone network and with the hydrophobic diterpene aglycones as the spacer sites that host water-insoluble drug molecules.

The new complexing agent diterpene glycosides (dTGs) have several advantages over CDs as complexing agents. First, dTGs may be less rigid on the requirement of the cavity size, which has been a limiting factor for the formation of (β-CDs-drug complexes, especially large molecular drugs. Second, the possible uniformity of hydrophilic-hydrophobic spacing alignment of dTGs may be more efficient than the circular hydrophilic-hydrophobic spacing alignment, and thus capable of solubilizing more drug molecules. Third, the dTGs have excellent water solubility and stability in water solution. The solubility of dTGs is 60 g/100 mL water at 25° C. and 80 g/100 mL water at 37° C. This is much higher than β-CD of 1.85 g/100 mL water, α-CD of 15 g/100 mL water, or γ-CD of 23 g/100 mL water. In water solutions, dTGs were structurally stable for months. Fourth, pH stability of the diterpene glycosides used as complexing agents range from 1.5 to 11, a much wider pH range than the CDs. Fifth, the diterpene glycosides may actually be safer for internal injections. Some diterpene glycosides have been approved by the FDA as sweeteners (e.g., rebaudioside A). Based on the aglycone steviol, estimates are that daily consumption of steviol glycosides of 8 mg rubusoside/kg body weight is safe and has no adverse effect, and up to 766 mg rubusoside/kg body weight (based on 383 mg/kg body weight daily expressed as steviol) is the no-observed-effect level. The intraperitoneal injection of stevioside water solution in hypertensive rats at doses of 50 mg/kg and 100 mg/kg body weight showed no adverse effects (Y.-H. Hsu et al., Antihypertensive Effect of Stevioside in Different Strains of Hypertensive Rats. Chinese Medical Journal (Taipei) 2002; vol. 65:1-6). In pharmaceutical dosing paradigm, 50 mg/kg or less of rubusoside may be sufficient to solubilize drugs to therapeutic levels for parental applications. Additionally, the geometry of diterpene glycosides as complexing agents to increase solubility of water-insoluble drugs may increase bioavailability by readily exposing the drug molecules to the bi-layer membranes of the target cells for rapid absorption. Moreover, the formed rubusoside-curcumin complexes in water solutions were shown resistant to heat up to 115° C. and pH changes from acid to alkaline conditions. Last, the heat stability of diterpene glycosides up to 250° C. allows effective use of melting and other heating methods in the preparation of solid complexes. Based on the above comparisons, features, and experimental data shown in this invention, it is believed that the dTGs are superior to CDs as complexing agents in the solubilization of water-insoluble drugs.

Using the diterpene glycosides as solubilizers provides a way to alleviate problems with low solubility drugs, e.g., low absorption and low bio-availability of the drug. In addition, using the solubilizer and drug in a powder form (containing solubilizer-drug complexes) will allow solid formulations that are readily dissolvable in water, e.g., tablet or even effervescent tablets. The solubilizers can be used to prepare non-alcoholic syrups of low solubility drugs that are stable, or to prepare gelatin capsules with the solubilizer and drug inside.

The solubilizer and solubilized drug may be administered to a patient by any suitable means, including orally, parenteral, subcutaneous, intrapulmonary, topically (e.g., ocular or dermal), rectal and intranasal administration. Parenteral infusions include intramuscular, intravenous, intraarterial, or intraperitoneal administration. The solution or its dry ingredients (containing solubilizer-drug complexes) may also be administered transdermally, for example in the form of a slow-release subcutaneous implant, or orally in the form of capsules, powders, or granules.

Pharmaceutically acceptable carrier preparations for parenteral administration include sterile, aqueous or non-aqueous solutions, suspensions, and emulsions. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. The solubilizer and drug may be mixed with other excipients that are pharmaceutically acceptable and are compatible with the active ingredient in the drug. Suitable excipients include water, saline, dextrose, glycerol and ethanol, or combinations thereof. Intravenous vehicles include fluid and nutrient replenishers, electrolyte replenishers, such as those based on Ringer's dextrose, and the like. Preservatives and other additives may also be present such as, for example, antimicrobials, anti-oxidants, chelating agents, inert gases, and the like.

The form may vary depending upon the route of administration. For example, compositions for injection may be provided in the form of an ampule, each containing a unit dose amount, or in the form of a container containing multiple doses.

For purposes of this application, a compound that is insoluble in water is a compound in which less than 100 μg dissolves in 1 mL water. A compound that is sparingly soluble in water is one in which less than 20 mg, but more than 100 μg, dissolves in 1 mL water. Finally, in general, a compound that has low solubility in water is one in which less than 20 mg dissolves in 1 mL water.

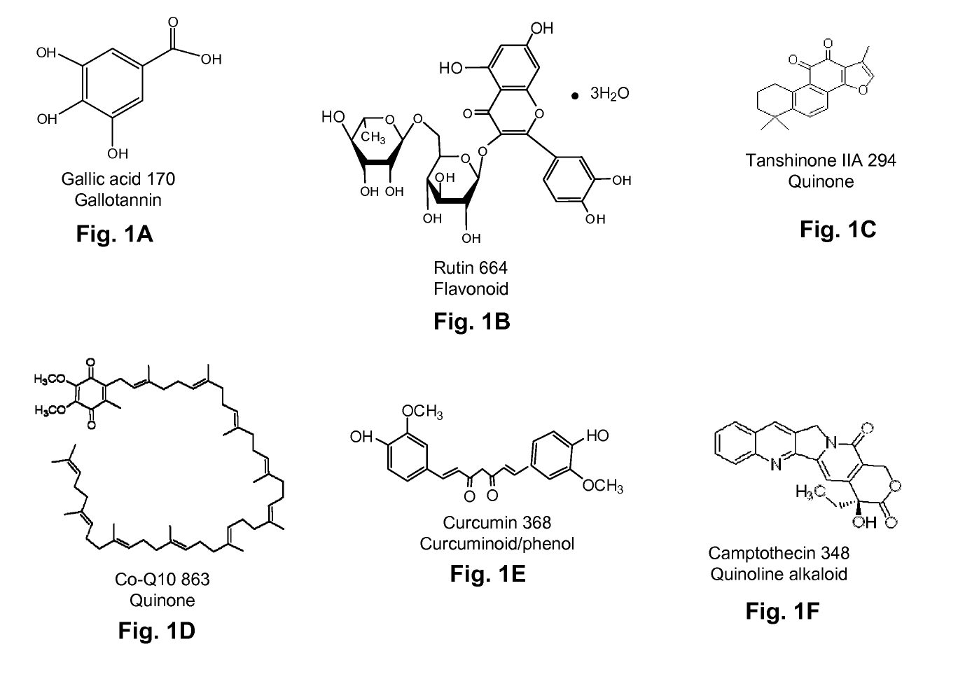

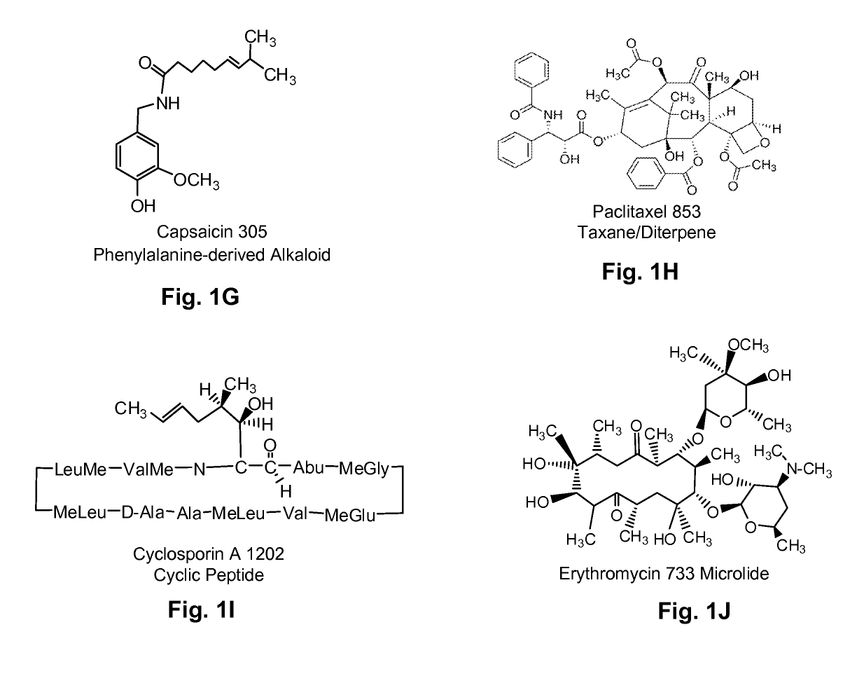

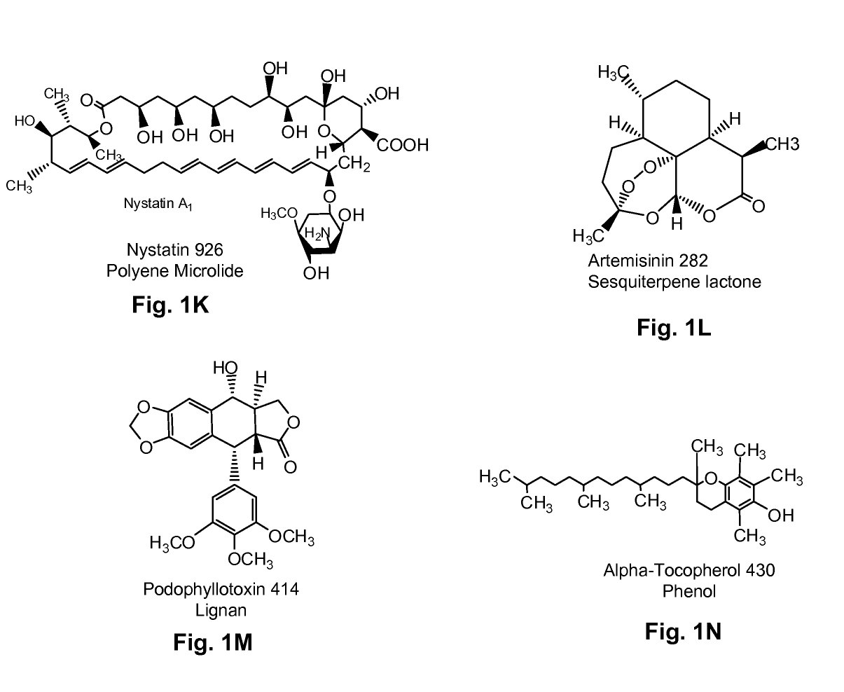

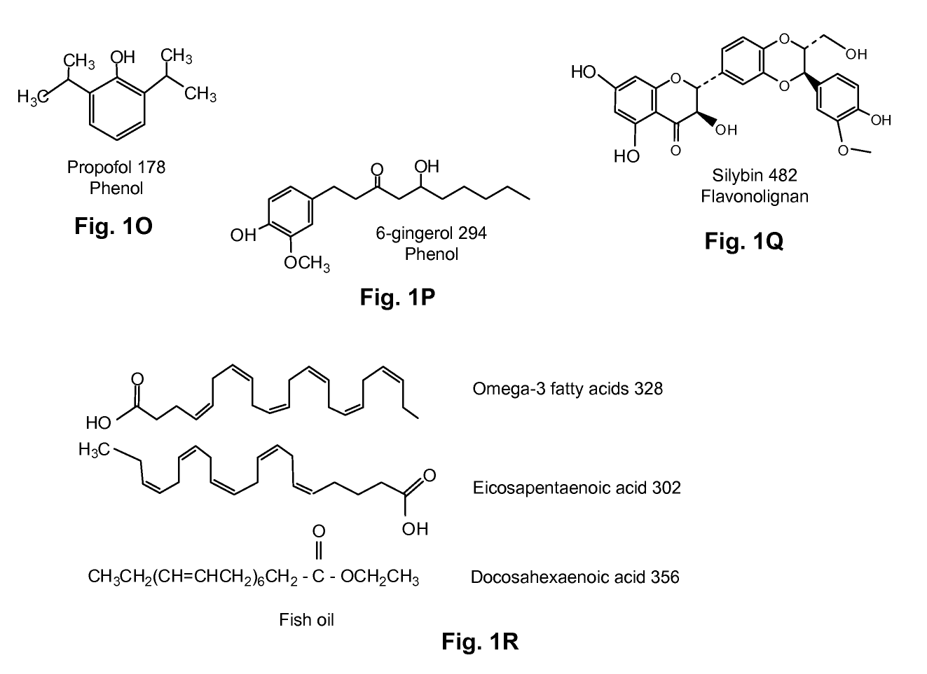

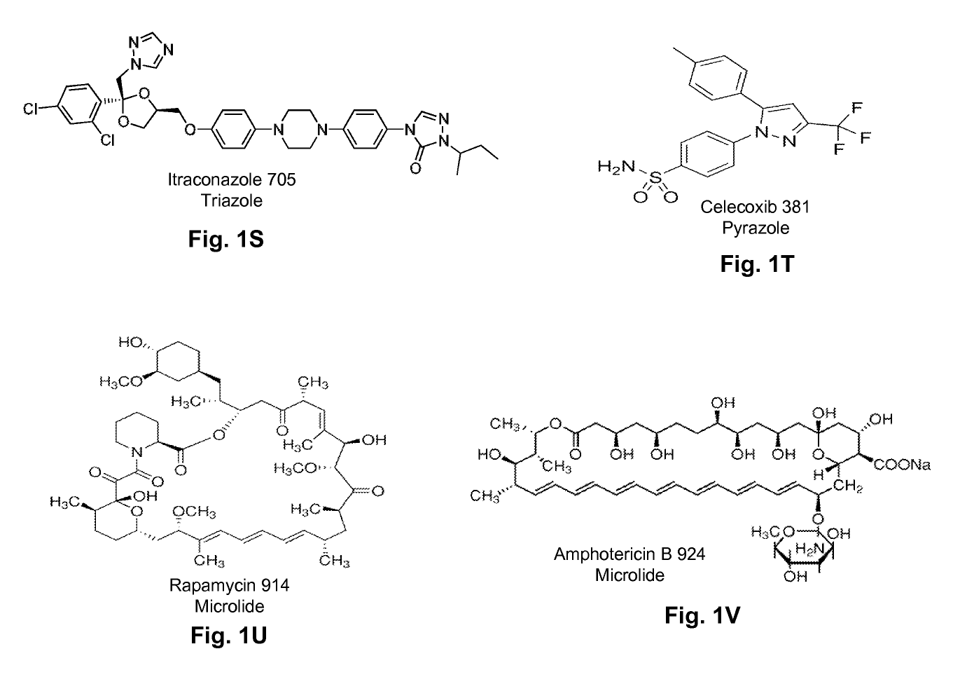

The structures of representative compounds of the various classes of organic compounds with low solubility are shown in FIGS. 1A to 1V. A summary of some of the experimental data using these compounds and a natural diterpene glycoside, rubuososide, is given in Table 1. The details of these experiments, including results from control experiments, are given below:

TABLE 1
Pharmaceutical/Bioactive Compounds in Water Solutions Containing
a Natural Diterpene Glycoside Solubilizer (Rubusoside)

| | | | | Solubility in | Solubility in | |
|---|---|---|---|---|---|---|
| | | | Natural | water¹ without | water with | Solubility |
| | | | Solubilizer | solubilizer | solubilizer | increase |
| Compounds | M.W. | Class | (% w/v) | (μg/mL) | (μg/mL) | factor |
| Gallic acid | 170 | Hydrolysable tannin | 40 | 11,000 | 106,000 | 9.6 |
| Rutin | 610 | Flavonoid | 10 | 125 | 1750 | 14 |
| Curcumin | 348 | Curcuminoid/phenol | 5 | 0.6 | 171 | 285 |
| Tanshinone IIA | 294 | Quinone | 20 | 0.01 | 127 | 12700 |
| Coenzyme Q10 | 863 | Quinone | 10 | 0.1 | 111 | 1110 |
| Capsaicin | 305 | Phenylalanine-derived | 10 | 57 | 4,920 | 86 |
| | | Alkaloid | | | | |
| Camptothecin | 348 | Quinoline Alkaloid | 20 | 0.4 | 143 | 357 |
| Paclitaxel | 853 | Taxane/Diterpene | 20 | Not soluble | 232 | 662 |
| Amphotericin B | 924 | Polyene macrolides | 10 | 82 | 200 | 2.2 |
| Cyclosporine | 1202 | Cyclic peptides | 10 | 9 | 250 | 27 |
| Erythromycin | 733 | Macrolides | 10 | 459 | 5,333 | 11 |
| Rapamycin | 914 | Macrolides | 10 | 2.6 | 240 | 92 |
| (Sirolimus) | | | | | | |
| Nystatin | 926 | Polyene macrolide | 10 | 66 | 1,100 | 16 |
| Artemisinin | 282 | Sesquiterpene lactone | 10 | 55 | 280 | 5 |
| Podophyllotoxin | 414 | Lignan | 10 | 120 | 919 | 7.7 |
| Silybin | 482 | Flavonolignans | 10 | Poor | 150 | Many |
| (Silibinin) | | | | | | |
| 6-Gingerol | 294 | Phenol | 10 | Not soluble | 150 | Many |
| Propofol | 178 | Phenol | 10 | 158 | 11,700 | 74 |
| Alpha- | 430 | Phenol | 25 | Not soluble | 13,250 | Many |
| Tocopherol (VE) | | | | | | |
| Fish oil | n/a | lipid | 10 | Not soluble | Qualitative | Not |
| | | | | | | applicable |
| Itraconazole | 705 | Triazole | 10 | Not soluble | 21 | Many |
| Celecoxib | 381 | Pyrazole | 10 | Not soluble | 488 | Many |

FN 1. Solubility values are obtained from published Material Safety Data Sheets or The Merck Index, 1996.

### Example 1

### Materials and Methods

Sources of Solubilizers

Rubusoside: Rubusoside was extracted from Chinese sweet leaf tea leaves (Rubus suavissimus; Rosaceae) purchased from Natural Plants Products Factory, Guilin S&T New Tech Company, Sanlidian Campus of Guangxi Normal University, Guilin, Guangxi, China. Rubusoside has a molecular formula C₃₂H₅₀O₁₃ and molecular weight of 642.73. First, the air-dried leaves were boiled with water with a weight to volume ratio ranging from about 1:10 to about 1:20. From this extraction, a crude dried extract (20 to 30% dry weight yield from the raw leaves) was obtained that contained from about 5% to about 15% rubusoside by weight. The dried extract was then reconstituted with water to a weight to volume ratio ranging from about 1:4 to about 1:5. In this concentrated extract, the ellagitannins would partially precipitate out and were removed by filtration. The rubusoside was retained in the solution. The solution containing rubusoside was then subjected to column chromatography using a macroporous resin (Dowex Optipore L493 Polymeric Adsorbent, Styrene-Divinylbenzene polymers with 46 Angstrom average pore size; The Dow Chemical Company, Midland, Mich.). The column was eluted with ethanol to obtain a purified extract containing approximately 60% rubusoside and about 1% steviol monoside. Some of this extract was used in Example 16 below. Subsequently, the purified extract was loaded on a second column to further purify the extract using silica gel as the stationary absorbent (Silica Gel, 200-300 mesh, Natland International Corporation, Research Triangle, N.C.). The column was eluted with a mixed solvent (chloroform:methanol at a ratio of 8:2 v/v). The extract from this second column was at least 80% pure rubusoside, and was dried to a powder. Finally, this rubusoside-rich extract (>80% w/w) was dissolved in absolute methanol by heating to temperatures ranging from about 60° C. to about 80° C. The solution was then cooled to allow re-crystallization of rubusoside. This re-crystallization process may need to be repeated to obtain pure rubusoside (>99% purity as measured on HPLC). The structure of rubusoside was confirmed by mass spectrometry and NMR. Rubusoside, a diterpene glycoside, has a molecular weight of 642 Daltons, and is a white crystal or powder. The crystalline powder is stable at temperatures ranging from about −80° C. to over 100° C. In water, rubusoside itself has a solubility of approximately 400 mg/ml at 25° C. and 800 mg/ml at 37° C., which is greater than that of many common, water-soluble compounds (e.g., sodium chloride has a solubility of 360 mg/ml water).

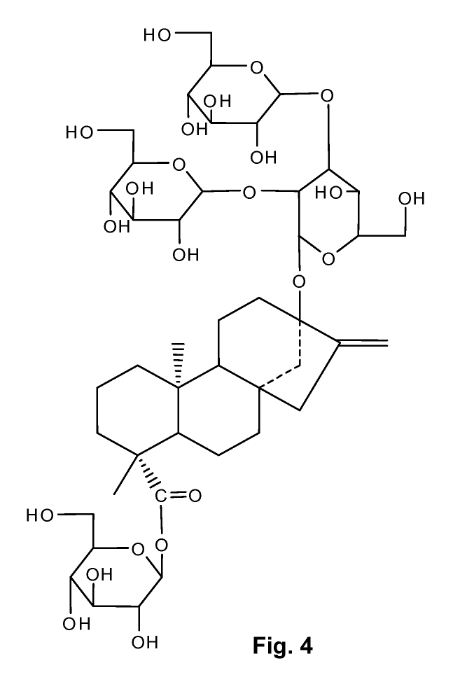

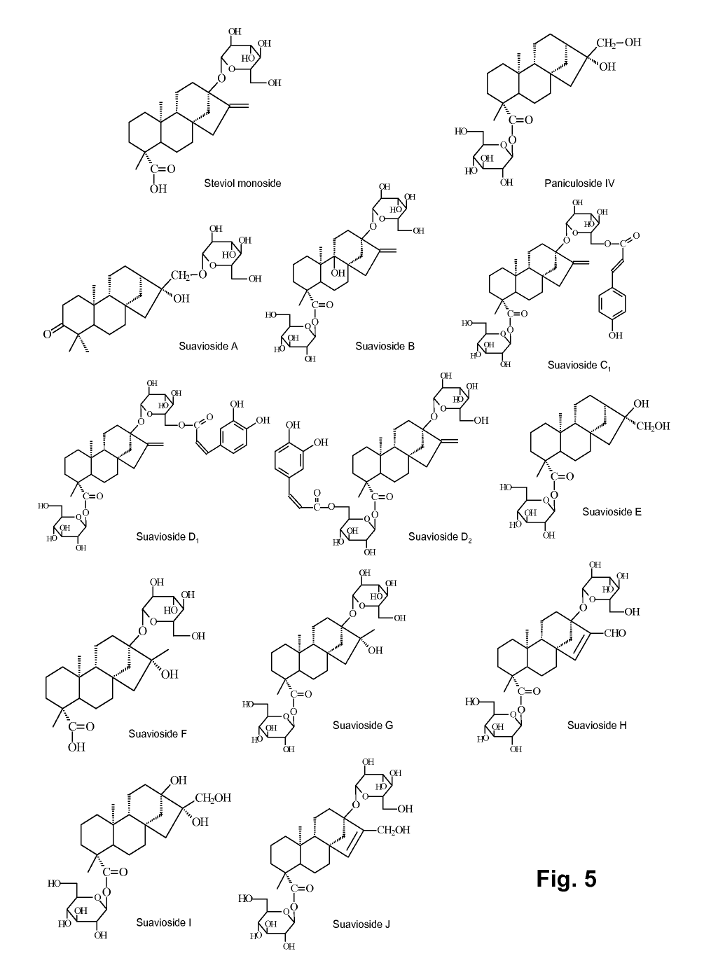

Stevioside: Stevioside is a diterpene glycoside that is isolated from the Stevia leaf (Stevia rebaudiana; Asteraceae). Stevioside has a molecular formula C₃₈H₆₀O₁₈ and a molecular weight of 804. The structure is shown in FIG. 4. Stevioside was purchased from Chromadex Inc. (Irvine, Calif.).

Rebaudioside A: Rebaudioside A is a diterpene glycoside that is isolated from the Stevia leaf (Stevia rebaudiana; Asteraceae). Its structure is shown in FIG. 5. Rebaudioside A was purchased from Chromadex Inc. (Irvine, Calif.).

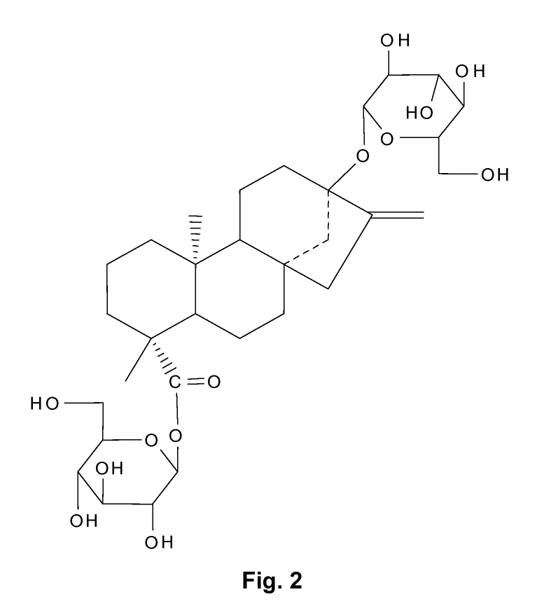

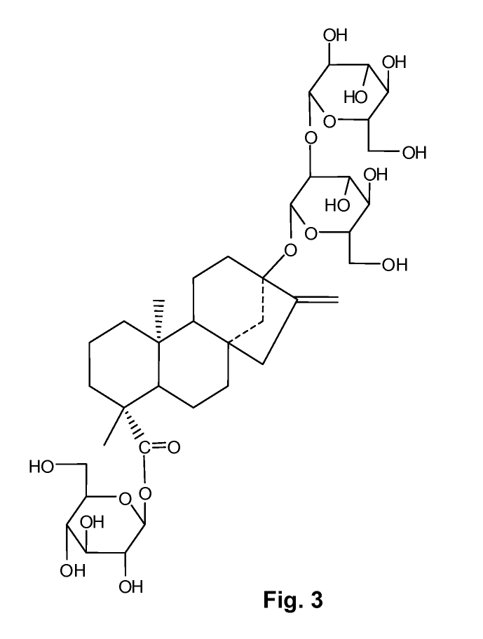

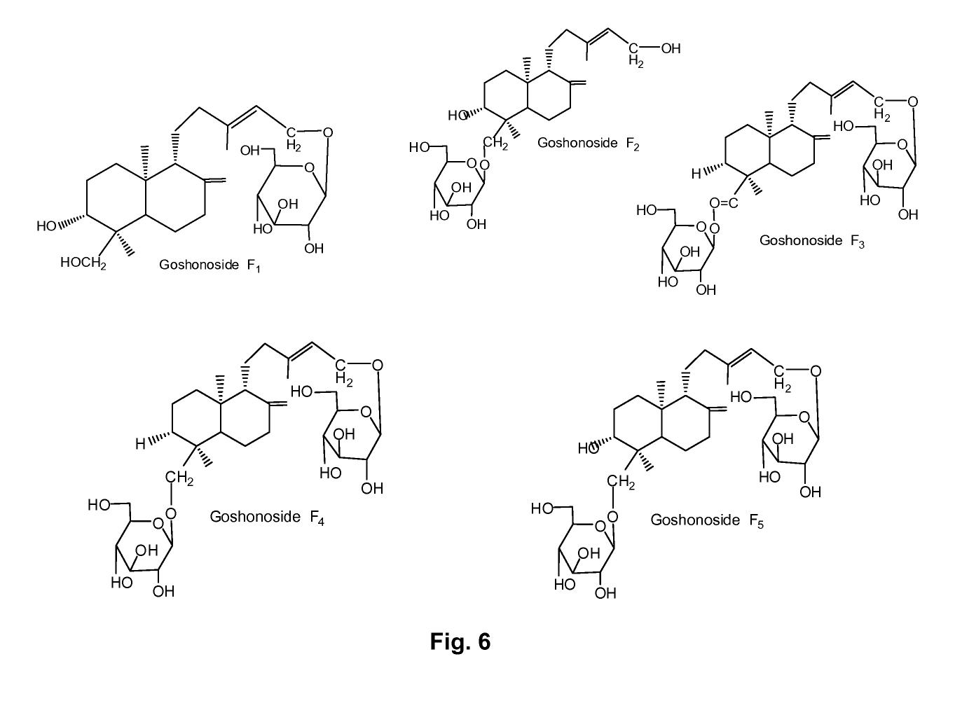

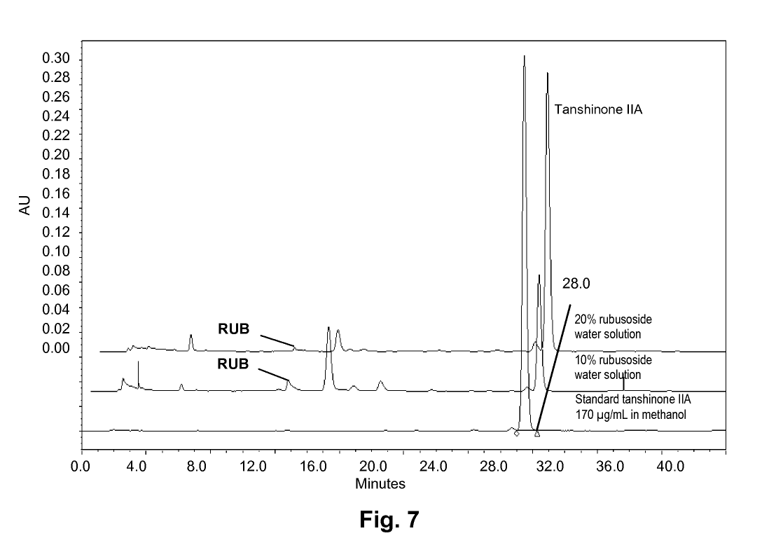

Steviol monoside: Steviol monoside is a diterpene glycoside that is isolated from the Chinese sweet leaf tea (Rubus suavissimus; Rosaceae), the same source as rubusoside. The structure of steviol monoside has only one glucose moiety (FIG. 6) rather than two as in rubusoside (FIG. 2). Steviol monoside can be isolated from the sweet leaf tea or be obtained through the acid hydrolysis of rubusoside to cleave one glucose unit.

Compounds Tested For Solubility: Twenty-two bioactive and pharmaceutical compounds with a water solubility ranging from poor (11 mg/ml) to nearly insoluble (0.01 μg/ml) were used. All compounds were found to have a purity greater than 98% based on HPLC (unless otherwise indicated). Gallic acid was purchased from Sigma-Aldrich Chemicals (St. Louis, Mo.) and has poor solubility (11 mg/ml) (The Merck Index, 1996). Tanshinone IIA was purchased from Shanghai University of Traditional Chinese Medicine (Shanghai, China) and is nearly insoluble. Rutin, cucurmin, capsaicin, camptothecin, and paclitaxel were all purchased from Sigma-Aldrich Chemicals. Artemisinin, podophyllotoxin, silybin (Silibinin), and rapamycin (Sirolimus) were purchased from LKT Laboratories (St. Paul, Minn.). Gingerols were purchased from Chromadex Inc. (Irvine, Calif.). According to The Merck Index (1996), the solubilities of these compounds are as follows: rutin (nearly insoluble), curcumin (insoluble), capsaicin (very poor), camptothecin (insoluble), and paclitaxel (insoluble). According to the Material Safety Data Sheets, artemisinin, podophyllotoxin, silybin (Silibinin), and rapamycin (Sirolimus) have a water solubility of insoluble, nearly insoluble, poor, and insoluble, respectively. Gingerols are ginger oils that are not soluble in water

Three antifungal compounds (Amphotericin B, Cyclosporine, Nystatin) and an antibacterial compound (Erythromycin) were tested. These four antimicrobial compounds were purchased from Sigma-Aldrich Chemicals (St. Louis, Mo.) and are nearly insoluble. Moreover, three lipid soluble compounds were tested. Coenzyme Q10 was purchased from MP Biomedicals Inc. (Solon, Ohio). Alpha-tocopherol (vitamin E) and Propofol were purchased from Sigma-Aldrich Chemicals (St. Louis, Mo.). Fish oil was purchased from a local nutrition store (General Nutrition Center, Baton Rouge, La.). The water insoluble celecoxib was purchased from LC Laboratories (Woburn, Mass.); and another water insoluble compound itraconazole was purchased from LKT Laboratories (St. Paul, Minn.).

Solubility test methods: A compound with low solubility was selected and weighed into multiple centrifuge tubes. Each experimental tube received a known amount of the solubilizing agent being tested. The control tubes remained only with the compound. The same volume, 1 mL, unless otherwise indicated, of deionized and distilled water was added to each tube. Alternatively, a set percentage of water solutions containing the solubilizer to be tested (e.g., 5% w/v or 10% w/v) were prepared separately. In these cases, the solubilizer-water solutions were added directly to the tubes containing the low-solubility compound. The tubes were then vortexed briefly and then sonicated for 60 min at temperature of 50° C. unless otherwise indicated. After sonication, the tubes were placed on an orbital shaker at a speed of 80 rpm in an incubator set at 25° C. for at least 24 hr. The tubes were then centrifuged at 4000 rpm for 10 min. The supernatant solution was passed through a 0.2 μm or 0.45 μm filter and analyzed for the concentration of the low-solubility compound, and sometimes the solubilizing compound, by HPLC or UV-Vis spectrophotometer.

HPLC and UV-Vis Spectrophotometer Analysis: The solutions containing various compounds in the absence or presence of solubilizers were analyzed on HPLC which consisted of a solvent delivery pump unit, an autosampler (Waters 717 plus), a UV-Vis diode array detector (Waters 2996 Photodiode Array Detector, 190 to 800 nm) coupled with an EMD 1000 Mass Detector (Waters), and an evaporative light-scattering detector (Waters 2420 ELSD). The system was computer controlled, and the results were analyzed using Empower software. Calibrations curves were constructed using known concentrations of the compounds and were used to quantify the concentrations of the compounds dissolved in solution. A more complete description of the HPLC procedure for some of the diterpene glycosides is found in G. Chou et al., “Quantitative and fingerprint analyses of Chinese sweet tea plant (Rubus suavissimus S. Lee),” J. Agric. Food Chem., vol. 57, pp. 1076-83 (2009).

Rutin was analyzed on a UV-Vis spectrophotometer (Beckmann Instruments) at a wavelength of 411 nm. The ratio of peak areas was used to calculate the increase in water solubility in the absence or presence of solubilizers.

### Example 2

### Effect of Rubusoside on the Water Solubility of Rutin

Rutin, a light-yellow colored compound, is a potent anti-oxidant that inhibits some cancers and reduces symptoms of haemophilia. It is known to have poor solubility in water (Table 1; 125 μg/ml; The Merck Index, 1996). In the presence of 100 mg rubusoside, 14-fold more rutin went into the aqueous solution, thus increasing the solubility of rutin to approximately 1.75 mg/ml (Table 2).

TABLE 2
Rutin solubility in the presence of rubusoside (RUB)

| | | Solubility |
|---|---|---|
| | Absorption | increase |
| Complex | (411 nm) | factor |
| Rutin | 0.281 | 1 |
| Rutin-RUB | 3.086 | 14 |

### Example 3

### Effect of Rubusoside on the Water Solubility of Tanshinone II

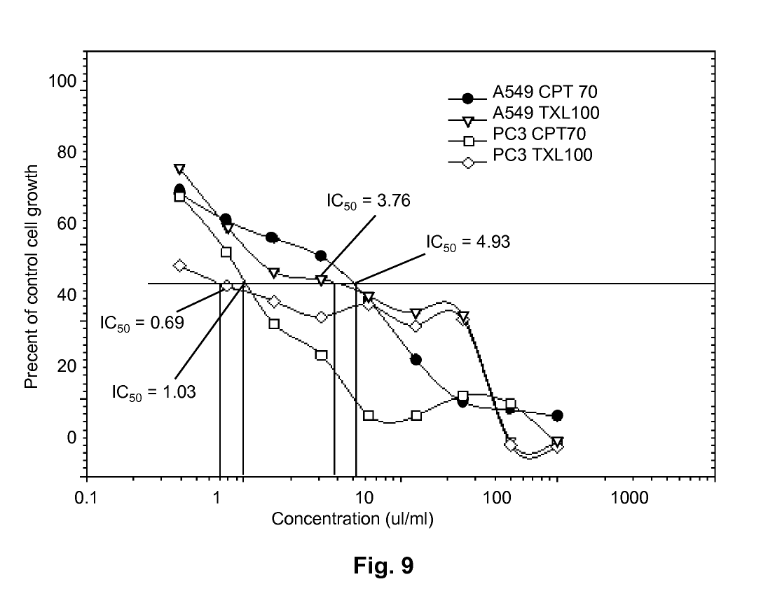

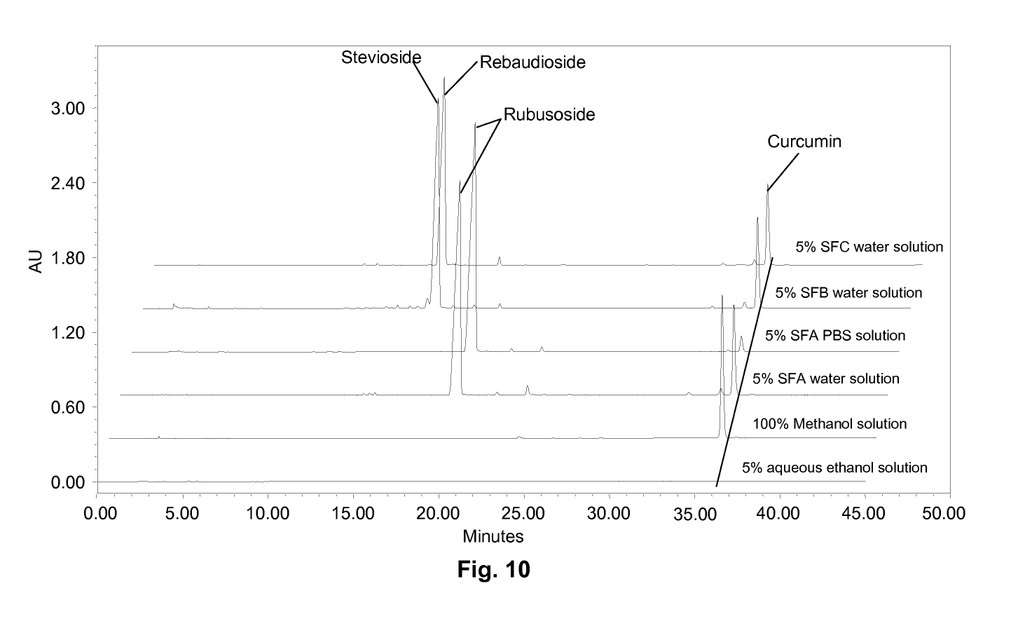

Tanshinone IIA is one of the natural analogues of tanshinone. Tanshinone IIA (as well as other tanshinones such as tanshinone I, dihydrotanshinone, and cryptotanshinone) is soluble in methanol but insoluble in water. In the presence of a 100 mg/ml concentration of rubusoside (10% w/v), tanshinone IIA went into solution. The concentration was measured using HPLC at a wavelength of 281 nm with the elution of tanshinone IIA at about 27.50 min. The concentration of tanshinone IIA in 100 mg/ml rubusoside was about 53.28 μg/ml (FIG. 9, middle chromatogram). In the presence of a 200 mg/ml concentration of rubusoside (20% w/v), tanshinone HA concentration in solution was about 127.72 μg/ml. (FIG. 9, upper chromatogram) Without the presence of rubusoside but using absolute methanol as a solvent, a standard tanshinone IIA solution was made to about 170 μg/ml (FIG. 9, lower chromatogram).

### Example 4

### Effect of Rubusoside on the Water Solubility of Gallic Acid

Gallic acid is reported to be both an antioxidant and antiangiogenic agent (See, for example, Published International Application WO 2005/000330). Gallic acid has low solubility (11 mg/ml) in water at room temperature, and the solution is light sensitive (The Merck Index, 1996). The gallic acid-water solution turned green within hours. At a 1:1 molar ratio or 1:3.77 weight ratio of gallic acid: rubusoside, the amount of gallic acid dissolved in solution increased with increasing amounts of rubusoside. For example, at 25° C., 106 mg gallic acid was dissolved in 1 ml water in the presence of 400 mg rubusoside, a 9.6 fold increase over the amount of gallic acid dissolved in plain water. At 37° C., 212 mg gallic acid was dissolved in 1 ml water in the presence of 800 mg rubusoside, a 19.3 fold increase. In addition, the gallic acid-rubusoside solution remained clear for several days longer than a solution of only gallic acid-water before gradually turning to greenish color, indicating some increase in photo-stability.

### Example 5

### Effect of Rubusoside on the Water Solubility of Curcumin

Curcumin is an orange-yellow pigment that is found in the rhizome of Curcuma longa, the source of the spice turmeric. Curcumin is a lipophilic compound that is insoluble in water (Table 1; The Merck Index, 1996). When added to water, the solution remains clear and colorless. However, in the presence of rubusoside (100 mg/ml), the orange-yellow curcumin dissolves and turns the solution an orange color (at pH greater than 7.0) or yellow color (at acidic pH below 7.0). HPLC analysis showed that 116 μg curcumin was dissolved in 1 ml water in the presence of 100 mg rubusoside (Table 3), a 193 fold increase in water solubility.

TABLE 3
Curcumin solubility in the presence of rubusoside (RUB).

| | μg/ml | Solubility |
|---|---|---|
| | (HPLC-UV | increase |
| Complex | at 426 nm) | factor |
| Curcumin | 0.6 | 1 |
| Curcumin-RUB | 116 | 193 |

In another experiment, a 10% w/v rubusoside water solution was prepared first. Ten milliliters of the rubusoside solution was added to 10 mg of curcumin (Cayman Chemical, Ann Arbor, Mich.), mixed and sonicated for 60 min at 60° C. This solution was then autoclaved at 115° C. and 1 atmosphere pressure for 30 min. The autoclaved solution was in an incubator at 37° C. for 72 hr. The solution had minimum light exposure at all times. The solution was then filtered through a 0.45 μm Nylon filter and analyzed by HPLC analysis as previously described. Curcumin in this solution was 462 μg/ml. The higher concentration was possibly due to the additional heating step.

In another experiment, a 5% w/v rubusoside water solution was prepared first. Ten milliliters of the rubusoside solution was added to 10 mg of curcumin (Cayman Chemical, Ann Arbor, Mich.), mixed and sonicated for 60 min at 60° C. This solution was then autoclaved at 115° C. and 1 atmosphere pressure for 30 min. The autoclaved solution was in an incubator at 37° C. for 72 hr. The solution had minimum light exposure at all times. The solution was then filtered through a 0.45 μm Nylon filter and analyzed by HPLC analysis as previously described. Curcumin in this solution was 171 μg/ml, an increase in solubility of 285-fold.

### Example 6

### Effect of Rubusoside on the Water Solubility of Camptothecin

Camptothecin (CPT) is a cytotoxic alkaloid that was first isolated from Camptotheca accuminata. It has a poor solubility in water (Table 1; The Merck Index, 1996). In the presence of rubusoside, CPT was soluble in water. Concentrations of CPT in the aqueous solutions were measured using HPLC. Using 70 mg (109 mM) rubusoside, 50 μg/ml (0.144 mM) CPT was dissolved in solution. (Table 4) The molar ratio of CPT:rubusoside in this solution was 1:757 and the weight ratio was 1:1400. This solution was stable at room temperature for at least two weeks. As shown in Table 3, a higher solubility of CPT (143.7 μg/ml) was obtained when using 200 mg rubusoside.

TABLE 4
Camptothecin (CPT) solubility in the presence of
rubusoside (RUB).

| | Solubility | Solubility |
|---|---|---|
| | of CPT | increase |
| Complex | (μg/ml) | factor |
| CPT | 0.4 | 1 |
| CPT + 70 mg RUB | 50.3 | 125.75 |
| CPT + 200 mg RUB | 143.7 | 359.25 |

### Example 7

### Effect of Rubusoside on the Water Solubility of Capsaicin

Capsaicin (CAP) is a pungent phenylalanine alkaloid derived from chili peppers and is practically insoluble in cold water (Table 1; The Merck Index, 1996). In the presence of rubusoside, however, capsaicin dissolved in water in increasing amounts as the amount of rubusoside increased. Capsaicin alone dissolved in water only at a concentration of 57 μg/ml (Table 5). In the presence of rubusoside, the amount of dissolved capsaicin in 1 ml water increased to 589 μg and 4920 μg in the presence of 20 mg and 100 mg rubusoside, respectively.

TABLE 5
Capsaicin (CAP) solubility in the presence of rubusoside (RUB)

| Complex | Solubility (μg/ml) | Solubility increase factor |
|---|---|---|
| CAP + 0 RUB | 57 | 1 |
| CAP + 20 mg RUB | 589 | 10 |
| CAP + 100 mg RUB | 4920* | 86 |
| CAP + 200 mg RUB | 4920* | >86 |
| CAP + 400 mg RUB | 4920* | >86 |

*The total amount of capsaicin in solution was 5000 μg.

### Example 8

### Effect of Rubusoside on the Water Solubility of Paclitaxel

Paclitaxel is a known anti-cancer diterpenoid alkaloid that is insoluble in water. (Table 1; The Merck Index, 1996) In the presence of rubusoside, however, paclitaxel dissolved in water in increasing amounts as the concentration of rubusoside increased, without any other additives. Paclitaxel was detectable in water solution when 20 mg rubusoside was present (0.35 μg/ml; Table 6). In the presence of 100 mg, 200 mg, and 400 mg rubusoside, the amount of paclitaxel dissolved in 1 ml water was 10 μg, 232 μg, and 351 μg, respectively. Thus, the solubility of paclitaxel increased up to 877 fold in the presence of rubusoside. The solution was stable at room temperature for at least two weeks.

TABLE 6
Paclitaxel (Taxol) solubility in the presence of rubusoside (RUB)

| Complex | Solubility (μg/ml) | Solubility increase factor |
|---|---|---|
| Taxol + 20 mg RUB | 0.35 | 1 |
| Taxol + 100 mg RUB | 10 | 29 |
| Taxol + 200 mg RUB | 232 | 662 |
| Taxol + 400 mg RUB | 351 | 1003 |

### Example 9

### Effect of Rubusoside on Cytotoxicity Against Cancer Cells by Paclitaxel and Camptothecin

Paclitaxel, a microtubule inhibitor, and camptothecin (CPT), a topoisomerase I inhibitor, have been widely used as chemotherapeutic agents. However, these agents have to be delivered either through a complex formulation to overcome poor solubility in the case of paclitaxel or in a modified structure in the case of CPT. The formulating components have created toxicities and side effects for paclitaxel thus limiting its therapeutic dose range; whereas, CPT itself has never been fully developed due to its poor solubility. Currently, paclitaxel (TAXOL®) injection is a clear, colorless to slightly yellow viscous solution that contains purified CREMOPHOR® EL (polyoxyethylated castor oil) and dehydrated alcohol (49.7%). CPT is given in the form of topotecan (a semi-synthetic derivative) hydrochloride solution containing inactive ingredients mannitol and tartaric acid. Rubusoside was used as a sole solubilizer to make aqueous solutions of both paclitaxel and CPT without any other additive components or co-solvents. The aqueous solution of each of the two drugs was clear, non-viscous, and stable in water solution.

Paclitaxel and camptothecin were purchased from Sigma Chemicals (St. Louis, Mo.). Both compounds had a purity of 95% or greater. To make an aqueous solution of paclitaxel, about 2 mg of paclitaxel was weighed into a solution containing 100 mg/ml rubusoside. The solution was sonicated for 60 min at 69° C. and then placed in a shaking incubator at 25° C. for 48 hr. The solution was then centrifuged at 4,000×g, and the supernatant was filtered with a 0.2 μm nylon filter. This aqueous solution was analyzed by HPLC and contained 17 μg/ml paclitaxel in the presence of 100 mg/ml rubusoside. The sample was labeled as TXL100. To make an aqueous solution of camptothecin, about 5 mg of camptothecin was weighed into a solution containing 70 mg/ml rubusoside. The solution was sonicated for 60 min at 69° C., and then placed in a shaking incubator at 25° C. for 48 hr. The solution was then centrifuged at 4,000×g and the supernatant was filtered with a 0.2 μm nylon filter. This aqueous solution was analyzed by HPLC and contained 10 μg/ml camptothecin in the presence of 70 mg/ml rubusoside. This sample was labeled as CPT70.

Inhibition of cellular proliferation was assessed by a MTT assay. Each well of the 96-well plate contained 10⁴ cells/100 μl cell culture growth medium. The cells were seeded onto the plate and placed in a 37° C. incubator overnight. On the next day, the compound treatment was set up. A series of dilutions using phosphate buffered saline (PBS) containing 10% FBS (fetal bovine serum) with either the test compound or vehicle with cell culture growth medium was made for each well with a final volume of 200 μl. Each treatment had 8 wells in a column. Prior to the addition of the test compound, the existing medium in each of the wells was aspirated completely and carefully to avoid losing cells. A series of dilutions of the aqueous samples were made ranging from 20 μl to 0.078 μl supplemented with culture media to make a final volume of 200 μl for each well. The plate was placed in a 37° C. incubator for 72 hr. On the day of the staining, MTT solution (3 mg Thiazolyl Blue Tetrazolium Bromide/ml PBS was prepared. To each well, 25 μl MTT solution was directly added. The plate was then incubated for 90 to 120 min. Afterwards, the wells were aspirated completely and carefully. Finally, 50 μl DMSO (dimethyl sulfoxide) was added to each well. The plate was read at 650 nm on a microplate reader. Cell proliferations were calculated as % of the control using the vehicle treatment as the control.

Calculations of IC₅₀ in molar concentrations were as follows. TXL: The sample concentration of TXL was 17 μg/ml=17 ng/μl, and the molecular weight of paclitaxel is 854. Thus, 1 μl/ml of TXL solution in the culture medium equaled 17 ng TXL/ml culture medium, which equaled 19.9 nM based on the molecular weight of 854. IC₅₀ values were expressed in 1 μl/ml and converted to molar concentrations based on the above conversion factor of 1 μl/ml=19.9 nM for paclitaxel. For example, IC₅₀ of 0.24 μl/ml=4.8 nM (PANC-1 TXL100), IC₅₀ of 0.69 μl/ml=13.7 nM (PC3 TXL100), and IC₅₀ of 3.76 μl/ml=74.8 nM (A549 TXL100).

CPT: The sample concentration of CPT was 10 μg/ml=10 ng/μl, and the molecular weight of camptothecin is 348. Thus, 1 μl/ml of CPT solution in the culture medium equaled 10 ng CPT/ml culture medium, which equaled 28.7 nM based on the molecular weight of 348. IC₅₀ values were expressed in μl/ml and converted to molar concentrations based on the above conversion factor of 1 μl/ml=28.7 nM for camptothecin. For examples, IC₅₀ of 1.91 μl/ml=54.9 nM (PANC-1 CPT70), IC₅₀ of 1.03 μl/ml=29.6 nM (PC3 CPT70), and IC₅₀ of 4.93 μl/ml=141.5 nM (A549 CTP70).

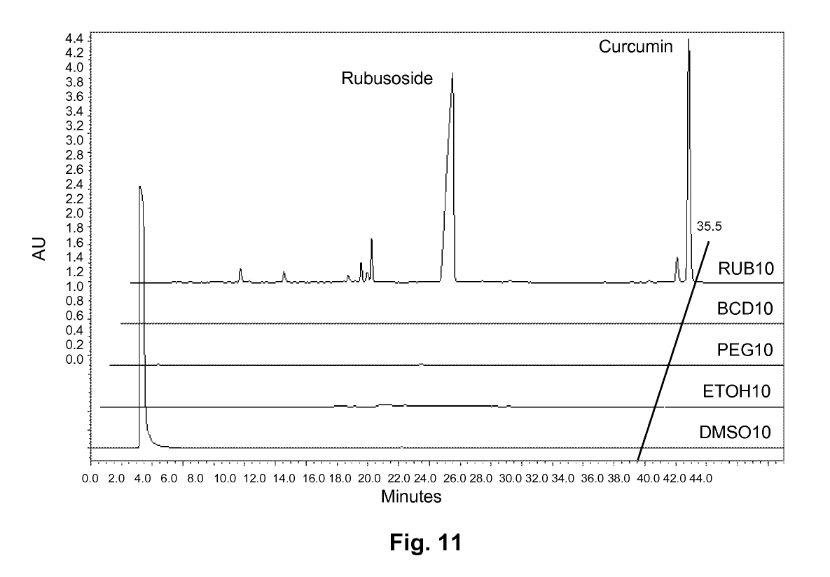

Aqueous solutions of paclitaxel and CPT were tested against three human cancer cell lines using MTT assays (FIGS. 10 and 11). All cancer cell lines were obtained from the American Type Culture Collection (ATCC), Manassas, Va. It was found that paclitaxel, at concentrations of 4.9 nM (0.24 μl/ml), 13.7 nM (0.69 μl/ml), or 74.8 nM (3.76 μl/ml), inhibited the proliferation of PANC-1 (human pancreatic), PC3 (human prostate), or A549 (human lung) cancer cells by 50%, respectively. CPT in its original structure displayed IC₅₀ values (50% inhibition) of 54.9 nM (1.91 μl/ml), 29.6 nM (1.03 μl/ml), or 141.5 nM (4.93 μl/ml) against PANC-1, PC3 or A549 cells, respectively.

These IC₅₀ values (mostly less than 0.1 μM) are significant for successful chemotherapeutic agents. Compared with reported IC₅₀ values for PC3 cells of 31.2 nM and 50 nM as anti-microtubule agent, this new formulation of paclitaxel, free of cremophor or alcohol, was twice as potent as the existing formulation. See, R. Danesi, et al., “Paclitaxel inhibits protein isoprenylation and induces apoptosis in PC-3 human prostate cancer cells.” Mol. Pharmacol. 1995 June; 47(6):1106-11; and E. K. Rowinsky, et al., “Taxol: A Novel Investigational Antimicrotubule Agent.” Journal of the National Cancer Institute, Vol. 82, No. 15, 1247-1259, 1990. Paclitaxel in its current drug formulation had to be greater than 1 μM to kill 50% of MDA-M231 breast cancer cells whereas other complicated formulations did not appear to improve this potency. See, A. O, Nornoo, et al., “Cremophor-free intravenous microemulsions for paclitaxel I: formulation, cytotoxicity and hemolysis.” Int J. Pharm. 2008 Feb. 12; 349(1-2):108-16.

The CPT solution was shown to be highly cytotoxic against all three cancer cell lines tested, especially potent against PANC-1. These IC₅₀ values fall in the lower end of the reported ones for CPT against various human cancer cell lines (10 nM to 3.5 μM). See, K. Kaczirek et al., “Cytotoxic Activity of Camptothecin and Paclitaxel in Newly Established Continuous Human Medullary Thyroid Carcinoma Cell Lines.” The Journal of Clinical Endocrinology & Metabolism Vol. 89, No. 5 2397-2401 (2004).

### Example 10

### Concentrations of Curcumin in Various Natural Solubilizers

A series of saturated water solutions of curcumin containing 5% (w/v) of various solubilizers were prepared as follows. First, approximately 2 mg of curcumin (reagent grade, Cayman Chemical Company, Ann Arbor, Mich.) was weighed into 1 mL water solutions each containing 5% w/v of rubusoside (isolated as described above), labeled as SFA; 5% stevioside (ChromaDex; Irvine, Calif.) labeled as SFB; or 5% rebaudioside A (ChromaDex, Irvine, Calif.), labeled as SFC. After sonication at 60° C. for 60 min., the solutions were centrifuged at 4,000×g and filtered with 0.2 nylon filters prior to HPLC analysis. Approximately 2 mg of the same curcumin was weighed into a 5% w/v rubusoside solution in 1×PBS (HyClone Laboratories, Inc., Logan, Utah). Approximately 2 mg of the same curcumin was weighed into a 5% v/v aqueous ethanol solution as a blank control. Additionally, a methanol solution at a concentration of 216 μg/mL of curcumin (analytical grade, ChromaDex) was prepared as a reference sample.

The HPLC conditions included the use of Prevail C18 column (4.6 mm×250 mm, 5 μm), a mobile phase consisting of acetonitrile (A) and 0.2% phosphoric acid in water (B) and running in gradient elution of 0-45 min at 20-80% A, dual detection wavelengths of 215 nm (for solubilizers) and 261 nm (for curcumin), and a flow rate at 1.0 mL·min⁻¹. Chromatograms were generated at the combined wavelengths of 215 nm and 261 nm shown in FIG. 10. Quantifications of curcumin were performed using the external standard calibration methods.

The 5% aqueous ethanol solution of curcumin (control; containing none of the solubilizers) showed only a baseline and not a single component that was detectable under the HPLC conditions (FIG. 10). The 100% methanol solution containing none of the solubilizers showed the curcumin to have a retention time of 36 min. The SFA solutions (water and PBS) showed curcumin at 36 min and rubusoside at 20 min whereas some other minor peaks came from the impurity of the curcumin compound (reagent grade claimed to be greater than 90% purity with the impure components as curcumin's natural analogues as curcuminoids). The SFB solution showed curcumin at 36 min and stevioside at 17 min whereas some other minor peaks came from the impurity of the curcumin. The SFC solution showed curcumin at 36 min and rebaudioside A slightly before 17 min whereas some other minor peaks came from the impurity of the curcumin.

Quantification of curcumin in each of the solutions indicated that 5% v/v aqueous ethanol solution did not dissolve any detectable curcumin into the solution, whereas the absolute methanol solution dissolved 216 μg/mL curcumin into the solution as prepared (Table 7). The 5% rubusoside solution pulled 136 μg/mL curcumin into the water solution whereas only 23.4 μg/mL curcumin was detected in the PBS water solution. The 5% stevioside water solution pulled 138 μg/mL curcumin into the water solution. The 5% rebaudioside A water solution pulled 122 μg/mL curcumin into the water solution. In term of solubilizing curcumin under the defined conditions, rubusoside and stevioside were equally and most effective; and rebaudioside A was slightly less effective. Using PBS solution caused reduction of curcumin concentration compared with the water solution without PBS.

TABLE 7
Concentrations of curcumin in various solutions containing solubilizing
natural compounds or alcohol solutions as measured using
HPLC analysis

| | CURCUMIN |
|---|---|
| CURCUMIN SOLUTION SAMPLES | CONCENTRATION μg/mL |
| 5% ethanol solution, no solubilizers | 0.0 |
| 5% rubusoside in water | 136.0 |
| 5% rubusoside in PBS solution | 23.4 |
| 5% stevioside in water | 138.0 |
| 5% rebaudioside A in water | 122.0 |
| 100% methanol standard; no solubilizers | 216.0 |

### Example 11

A. Steviol Glycosides as Solubilizers

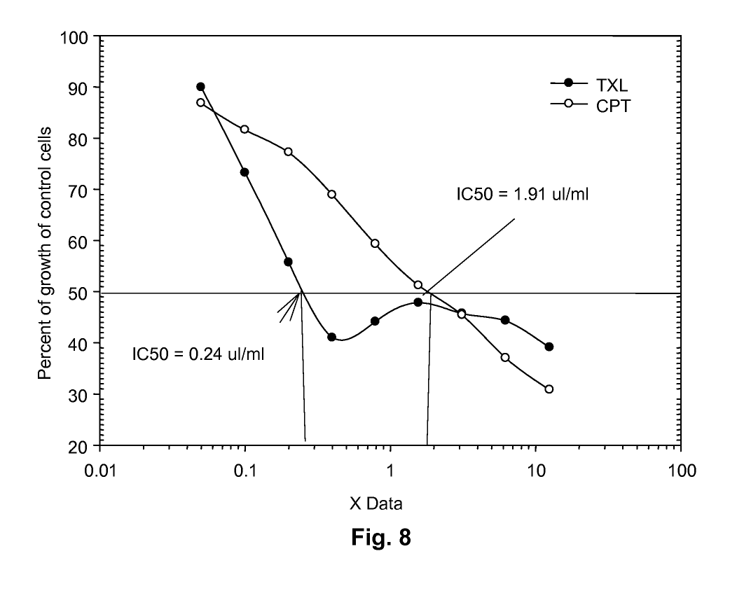

A series of saturated water solutions of curcumin containing 5% (w/v) of various steviol glycosides as solubilizers as shown in FIG. 5 will be prepared and analyzed in similar manner. The procedure will be as follows. First, approximately 2 mg of curcumin (reagent grade, Cayman Chemical Company, Ann Arbor, Mich.) will be weighed into 1 mL water solutions each containing 5% w/v of one of compounds in FIG. 8 such as steviol monoside, rebaudioside B, rebaudioside C, dulcoside A, steviolbioside, paniculoside IV, suavioside A, suavioside B, suavioside C1, suavioside D1, suavioside D2, suavioside E, suavioside F, suavioside G, suavioside H, suavioside I, and suavioside J. After sonication at 60° C. for 60 min., the solutions will be centrifuged at 4,000×g and filtered with 0.2 nylon filters prior to HPLC analysis. Approximately 2 mg of the same curcumin will be weighed into a 5% v/v aqueous ethanol solution as a blank control. Additionally, a methanol solution at a concentration of 216 μg/mL of curcumin (analytical grade, ChromaDex, Irvine, Calif.) will be prepared as a reference sample.

The HPLC conditions include the use of Prevail C18 column (4.6 mm×250 mm, 5 μm), a mobile phase consisting of acetonitrile (A) and 0.2% phosphoric acid in water (B) and running in gradient elution of 0-45 min at 20-80% A, dual detection wavelengths of 215 nm (for solubilizers) and 261 nm (for curcumin), and a flow rate at 1.0 mL·min⁻¹. Chromatograms are generated at the combined wavelengths of 215 nm and 261 nm. Quantifications of curcumin are performed using the external standard calibration methods.

B. Other Diterpene Glycosides as Solubilizers

A series of saturated water solutions of curcumin containing 5% (w/v) of various diterpene glycosides as solubilizers as shown in FIG. 6 as solubilizers will be prepared and analyzed in similar manner. The procedure will be as follows. First, approximately 2 mg of curcumin (reagent grade, Cayman Chemical Company, Ann Arbor, Mich.) will be weighed into 1 mL water solutions each containing 5% w/v of one of compounds in FIG. 9 such as goshonoside F₁, goshonoside F₂, goshonoside F₃, goshonoside F₄, and goshonoside F₅. After sonication at 60° C. for 60 min., the solutions will be centrifuged at 4,000 g and filtered with 0.2 nylon filters prior to HPLC analysis. Approximately 2 mg of the same curcumin will be weighed into a 5% v/v aqueous ethanol solution as a blank control. Additionally, a methanol solution at a concentration of 216 μg/mL of curcumin (analytical grade, ChromaDex, Irvine, Calif.) will be prepared as a reference sample.

The HPLC conditions include the use of Prevail C18 column (4.6 mm×250 mm, 5 μm), a mobile phase consisting of acetonitrile (A) and 0.2% phosphoric acid in water (B) and running in gradient elution of 0-45 min at 20-80% A, dual detection wavelengths of 215 nm (for solubilizers) and 261 nm (for curcumin), and a flow rate at 1.0 mL·min⁻¹. Chromatograms are generated at the combined wavelengths of 215 nm and 261 nm. Quantifications of curcumin are performed using the external standard calibration methods.

### Example 12

### Antifungal Agents in Water Solutions Containing a Natural Solubilizing Factor

Three widely used, water-insoluble antifungal agents, amphotericin B, cyclosporin A (also known as cyclosporine), and Nystatin (Sigma Chemical, St. Louis, Mo.), were selected for solubility testing using 10% w/v rubusoside water solution. A 10% w/v rubusoside (isolated as described above) water solution was prepared. Amphotericin B (2.0 mg), cyclosporin A (2.0 mg), and Nystatin (2.2 mg) were each weighed into centrifuge tubes. Then 10 mL, 7 mL, or 2 mL of the rubusoside solubilizing water solution were added to each compound in separate tubes. Each solution was sonicated at 50° C. for 60 min followed by incubation at 25° C. on a shaker in darkness for 12 hours.

All compounds appeared to go into solution completely. Amphotericin B solution contained 200 μg/ml in 10% solubilizing water solution; cyclosporin A water solution contained 250 μg/ml in 10% solubilizing water solution; and Nystatin water solution contained 1,100 μg/ml in 10% solubilizing water solution.

TABLE 8
Aqueous drug concentrations in 10% w/v
rubusoside (SFA) water solutions

| | AQUEOUS SOLUTION | 10% RUBUSOSIDE |
|---|---|---|
| COMPOUND | μg/ml | μg/ml |
| Amphotercin B | 82 | 200 |
| (yellow solution) | | |
| Cyclosporin A | 9 | 250 |
| (colorless solution) | | |
| Nystatin | 66 | 1,100 |
| (light-yellow solution) | | |

The above indicates that water solutions of amphotericin B and rubusoside are possible. This preparation would be virtually nontoxic, and could create new formulations to replace or complement the current liposomal formulation, for use orally, intravenously, and other modes of administration. The same will be true for cyclosporin and nystatin.

### Example 13

### Rubusoside as Solubilizer for Erythromycin

Erythromycin is poorly soluble in water, with a solubility of 459 μg/mL in water. To test if rubusoside can solubilize erythromycin in water, a 10% w/v rubusoside (isolated as described above) water solution was first prepared. Erythromycin (16.0 mg) was weighed into a centrifuge tube. Then 3 mL of the rubusoside solubilizing water solution was added to the compound. The solution was sonicated at 50° C. for 60 min followed by incubation at 25° C. on a shaker in darkness for 48 hours. Erythromycin completely dissolved in the water solution in the presence of 10% w/v rubusoside. This water solution was analyzed for erythromycin concentration using high performance liquid chromatography with detection of erythromycin at the wavelength of 410 nm, using the Luna C18 column (4.6 mm 250 mm, 5 μm) with the mobile phase consisted of acetonitrile (A) and 0.01M K₂HPO₄(B) in isocratic elution. The concentration of erythromycin in the rubusoside solution was 5.333 mg/mL.

### Example 14

### Comparison of Commonly Used Pharmaceutical Solvents and Excipients with Rubusoside in Solubilizing Curcumin

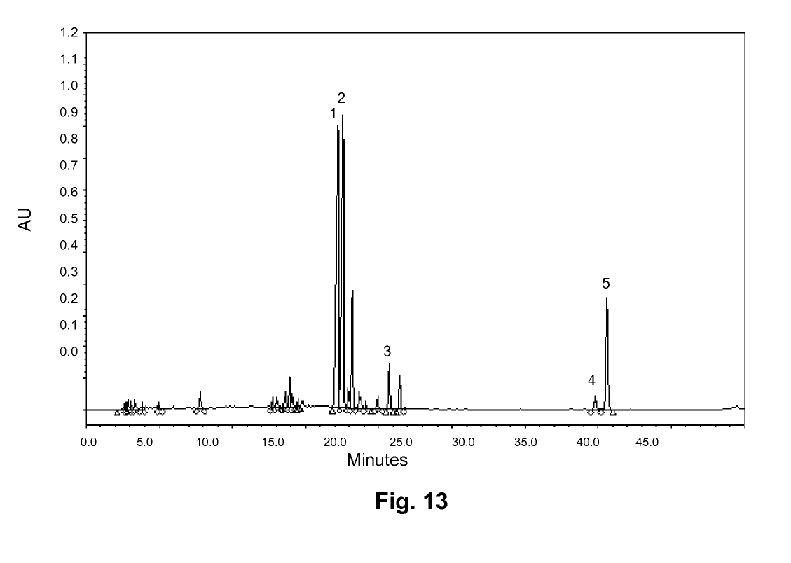

Solutions of 10% v/v aqueous ethanol (EtOH10), 10% v/v aqueous dimethyl sulfoxide (DMSO10), 10% polyethylene glycol 400 (PEG10), 10% w/v beta-cyclodextrin (BCD10), and 10% w/v rubusoside (RUB10) were prepared. Curcumin (approximately 2.0 mg) was weighed into each tube containing the various solubilizing solutions. After 60 min of sonication at 50° C., these solutions were incubated at 25° C. overnight. The solutions were analyzed for curcumin concentrations and the results are shown in FIG. 11. The HPLC analyses were conducted at wavelengths of 215 nm and 425 nm. A Prevail C18 column (4.6 mm×250 mm, 5 μm) was used, and the mobile phase consisted of acetonitrile (A) and 0.2% phosphoric acid (B). Under these conditions, curcumin was eluted at about 35.50 min. As shown in FIG. 13, the only solution with detectable curcumin was the 10% rubusoside solution, which contained about 232 μg/ml curcumin. This difference in the rubusoside-curcumin complex and (β-CD-curcumin complex (no detectable curcumin) in the same weight/volume ratio may be explained by the difference in water solubility of rubusoside (about 60 g/100 mL water) and β-CD (1.85 g/100 mL water).

### Example 15

### Solubility of Curcumin in the Presence of a Mixture of Steviol Glycosides

1. Sweet Leaf Tea Extract Containing Steviol Glycosides.

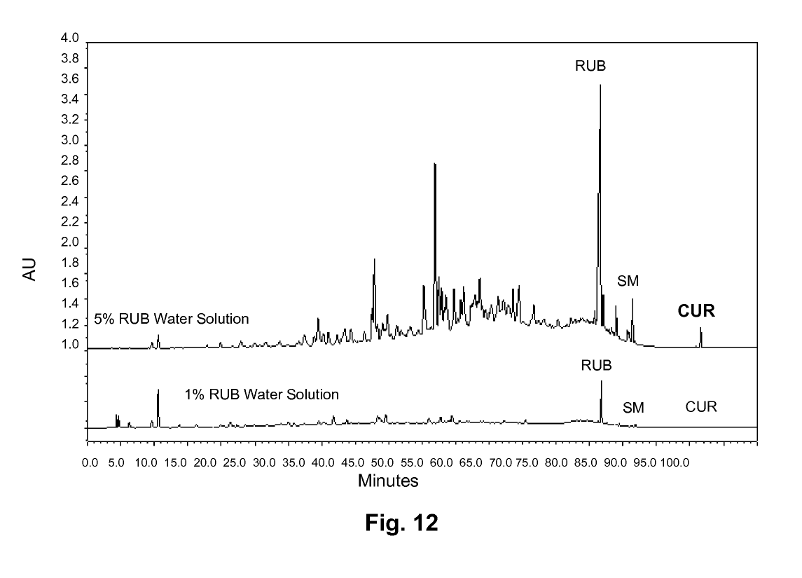

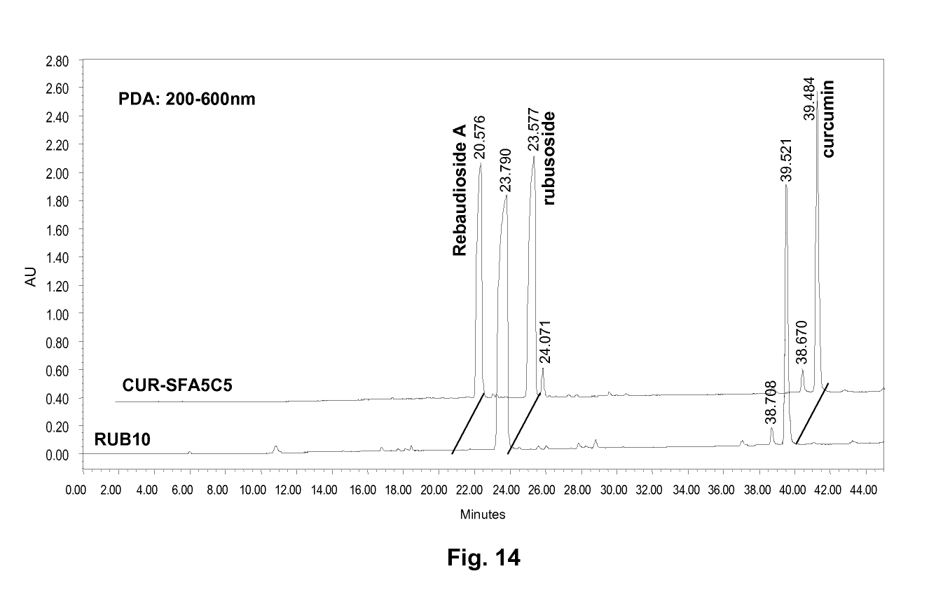

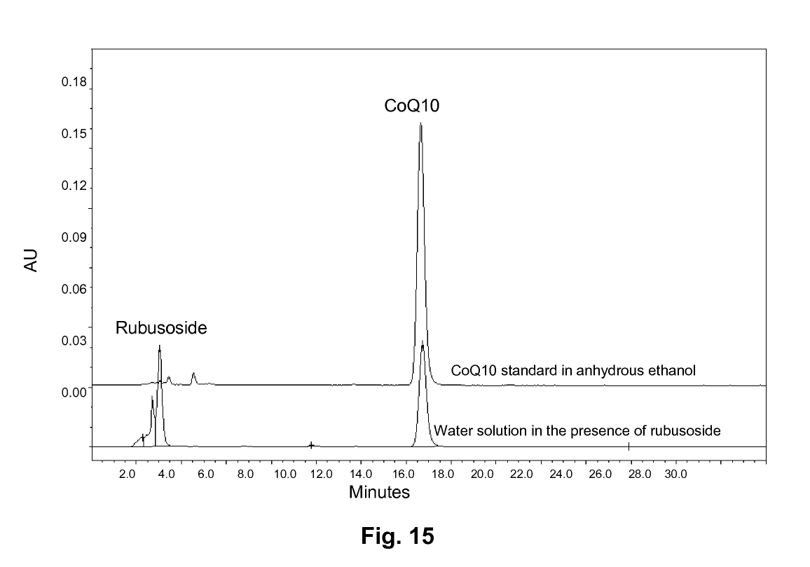

A solution (5 ml) of 8.62% v/v aqueous sweet leaf tea extract composed of 58% w/w rubusoside and approximately 1% w/w steviol monoside (prepared as described in Example 1) was prepared. The final solution contained 5% v/v rubusoside. Curcumin (approximately 2.0 mg) was weighed into a tube containing the 5% rubusoside solubilizing solution. After 60 min of sonication at 50° C., this solution was incubated at 25° C. overnight. This 5% solution and a 1% solution (4:1 water:5% RUB solution) were analyzed for curcumin concentrations on HPLC, and the results are shown in FIG. 12. The HPLC analyses were conducted at wavelengths of 215 nm and 425 nm. A Prevail C18 column (4.6 mm×250 mm, 5 μm) was used, and the mobile phase consisted of acetonitrile (A) and 0.2% phosphoric acid (B). Under these conditions, curcumin eluted at about 35.50 min. In FIG. 14, the rubusoside peak is indicated by RUB, the steviol monoside peak by SM, and the curcumin peak by CUR. In the 5% RUB (rubusoside) solution, curcumin concentration was measured to be about 51 μg/ml. Whereas in the 1% solution, curcumin was negligible.

2. Stevia Leaf Extract Containing about 5% w/v of a Mixture of Steviol Glycosides.

An extract of stevia leaf was purchased (Smarter Health Corporation, Jacksonville, Fla.), and its composition measured using HPLC as described above. A solution was made from this extract to contain a 5% w/v mixture of steviol glycosides, comprising of 55.80% w/w rebaudioside A, 43.42% w/w stevioside, 0.75% w/w rubusoside, and 0.04% w/w steviol monoside. Curcumin (approximately 2.0 mg) was weighed into a tube containing the 5% mixture of solubilizing steviol glycosides. After 60 min of sonication at 50 C, the solution was incubated at 25° C. overnight. This solution was analyzed for curcumin concentration on HPLC. The HPLC analyses were conducted at wavelengths of 215 nm and 425 nm. A Prevail C18 column (4.6 mm×250 mm, 5 μm) was used, and the mobile phase consisted of acetonitrile (A) and 0.2% phosphoric acid (B). Under these conditions as shown in FIG. 13, the following compounds eluted at the relative times: compound 1: rebaudioside A (17.212 min); compound 2: stevioside (17.557 min); compound 3: rubusoside (20.721 min); compound 4: demethyosycurcumin (34.807 min); and compound 5: curcumin (35.592 min). In the 5% mixed steviol glycosides water solution, curcumin was detected to be about 51 μg/mL.

3. Rubusoside Versus a Mixture of Rubusoside and Rebaudioside A.

A 10% w/v rubusoside water solution was prepared as described above. Separately, a 10% w/v water solution of a mixture of rubusoside and rebaudioside A at 1:1 weight ratio was prepared. Ten milliliters of the rubusoside solution and of the mixture solution were added to two separate vials, each with 10 mg curcumin (Cayman Chemical, Ann Arbor, Mich.), mixed well, and sonicated for 60 min at 60° C. The two solutions were then autoclaved at 115° C. and 1 atmosphere pressure for 30 min. The autoclaved solutions were placed in an incubator at 37° C. for 72 hr. The solutions had minimum light exposure at all times. The solutions were then filtered through 0.45 μm Nylon filters and analyzed on HPLC, and the chromatograms were shown in FIG. 14. Curcumin in the 10% rubusoside water solution was 462 μg/ml and in the 10% mixture solution was 531 μg/ml. This indicates that a mixture of rubusoside and rebaudioside A solubilized a greater amount of curcumin than rubusoside by itself.

### Example 16

### Effect of Rubusoside on the Water Solubility of Coenzyme Q10, Fish Oil, and Propofol

A 10% w/v rubusoside (isolated as described above) water solution was prepared. CoQ10 (2.0 mg), Fish oil (10 mg), and Propofol (10 mg) were each weighed into separate centrifuge tubes. Then 10 mL of the rubusoside solubilizing water solution was added to each tube. Additional water samples without any solubilizers were prepared for fish oil and Propofol. The solutions were sonicated at 50° C. for 60 min followed by incubation at 25° C. for 72 hr. The CoQ10-rubusoside solution was measured using HPLC using a alcohol CoQ10 solution as the standard. Propofol-rubusoside water solution was analyzed on HPLC in comparison with a propofol methanol reference solution and a propofol water solution (without rubusoside). Fish oil-rubusoside water solution was analyzed on HPLC in comparison with a fish oil water solution (without rubusoside). FIG. 15 shows the chromatograms of a standard CoQ10-anhydrous ethanol solution and a CoQ10-rubusoside water solution (10% w/v rubusoside) detected at the wavelength of 275 nm. The Prevail C18 column (4.6 mm×250 mm, 5 μm) was used for the analyses. The mobile phase consisted of methanol (A) and absolute ethanol (B). CoQ10 eluted at 14.55 min, and rubusoside eluted at 2.75 min. The concentration of CoQ10 in the rubusoside water solution sample was 111.4 μg/mL.

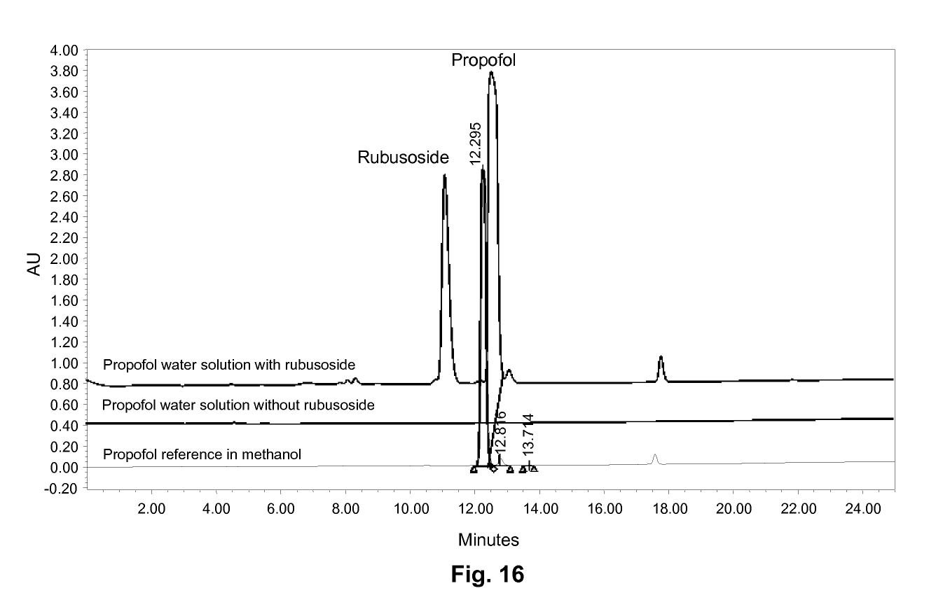

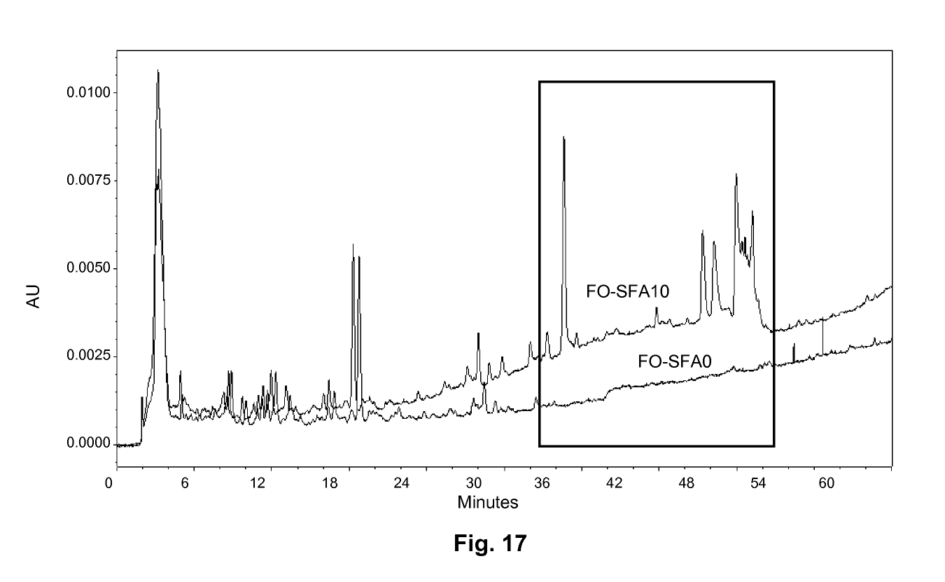

FIG. 16 shows the results of the HPLC analyses on propofol. The concentration of propofol in the water solution in the presence of 10% v/w rubusoside was 11.7 mg/mL or 1.17% w/v (FIG. 16). This was comparable to the propofol methanol solution. In contrast, the propofol water solution without the rubusoside had no measurable propofol. FIG. 17 shows the results of the fish oil solutions. The fish oil in the presence of 10% w/v rubusoside as a solubilizer showed more ingredients dissolved compared to the fish oil water sample that contained no rubusoside (FIG. 17). The box in FIG. 17 shows the difference in the two samples indicating that rubusoside pulled additional, unidentified components into water solution (FO-SFA10) as compared to the pure water solution with fish oil (FO-SFA0).

### Example 17

Effect of rubusoside on the water solubility of artemisinin, podophyllotoxin, alpha-tocopherol, silybin, rapamycin, and gingerols

A 10% w/v rubusoside (isolated as described above) water solution was prepared. Five milligrams of artemisinin, podophyllotoxin, silybin, rapamycin, or gingerols were weighed into separate centrifuge tubes. Then 5 mL of the rubusoside solubilizing water solution was added to each tube. The solutions were sonicated at 50° C. for 60 min followed by incubation at 25° C. for 72 hr. Separately, a 25% w/v stevia leaf extract (as described in Example 15) water solution was prepared. Five hundred milligrams of alpha-tocopherol were weighed into a centrifuge tube. Then 10 mL of the stevia leaf extract solubilizing water solution was added. The solution was sonicated at 50° C. for 60 min followed by incubation at 25° C. for 72 hr. These compounds in the solubilized water solutions were analyzed on HPLC and compared to a standard solution. In the presence of 10% w/v rubusoside, the aqueous solutions contained significant amounts of the tested compounds: 280 μg/mL artemisinin, 919 μg/mL podophyllotoxin, 150 μg/mL silybin, 240 μg/mL rapamycin, and 150 μg/mL 6-gingerol. In the presence of 25% w/v stevia leaf extract, 13,250 μg/mL alpha-tocopherol went into solution.

### Example 18

### Germicidal Activity of Curcumin in Rubusoside Water Solution

Germicidal activity of curcumin was determined by a modification of the AOAC. Germicidal and Detergent Sanitizer Test. The following challenge organisms were grown in trypticase soy broth for 24 h at 37° C.: Staphylococcus aureus (Gram-positive) ATCC 29740 (Newbould 305), Streptococcus agalactiae (Gram-positive) ATCC 27956 (McDonald 44), Streptococcus dysgalactiae (Gram-positive) ATCC 27957, Streptococcus uberis (Gram-positive) ATCC 27958, Escherichia coli (Gram-negative) ATCC 25922, Pseudomonas aeruginosa (Gram-negative) ATCC 27853, and clinical mastitis isolates of Enterobacter aerogenes (Gram-negative) (216RF) and Klebsiella pneumoniae (Gram-negative) (A37RR) from the Louisiana State University Hill Farm Research Station dairy herd.

Bacterial cultures. Aliquots of each 24-h bacterial culture were standardized to a turbidity of a 0.5 McFarland standard, which corresponds to approximately 150×10⁶ colony-forming units (cfu) per ml. Aliquots containing 0.02 ml of this culture were added to 1.98 ml aliquots of the curcumin-rubusoside solution. After 30 seconds and again after 10 min, 1 ml aliquots were removed from the combined microorganism/curcumin mixture and added to 9 ml of neutralizer (Letheen Broth, Difco Laboratories, Detroit, Mich., modified to contain 1% sodium thiosulfate). This solution was mixed thoroughly, and diluted 1:1000 in saline, and 0.1 ml was plated on duplicate Letheen Agar (Becton Dickinson, Cockeysville, Md.) plates for each microorganism tested. Resultant colonies were counted after incubation of the plates at 37° C. for 24 h.

First Experiment. The test compound was a solubilized curcumin water solution. For the first batch curcumin sample (Batch# CUR-SFA5-021209), 100 mL of a 5% w/v rubusoside water solution was prepared. This solubilizing solution was added to 24 mg of curcumin (Cayman Chemical, Ann Arbor, Mich.), mixed and sonicated for 60 min at 60° C. The solution was kept in the dark. The solution had a pH value of 6.5 and was filtered through a 0.45 μm Nylon filter and analyzed on HPLC. Curcumin in this solution was 158 μg/ml.

As a result of dilution, the actual pH of the curcumin solution in the culture medium was 6.1. In this experiment, inhibition of 99.99% and 43.75% growth of the Gram-Negative Pseudomonas aeruginosa and Streptococcus dysgalactiae took place within 30 seconds of co-culture with curcumin, and inhibition of 91.7% and 39.33% growth of the Gram-Positive Streptococcus agalactiae and Staphylococcus aureus occurred within 30 seconds of co-culture with curcumin. However, there was no observed inhibition of bacterial growth in the Gram-Positive Streptococcus uberis and Escherichia coli, and in two Gram-Negative Enterobacter aerogenes and Klebsiella pneumoniae (Table 9). In 10 min of co-culture, inhibition of growth was observed in these bacteria: Pseudomonas aeruginosa by 99.99%, Streptococcus agalactiae by 99.7%, Escherichia coli by 98.54%, Streptococcus dysgalactiae by 47.92%, and Staphylococcus aureus by 26.67%. There was no growth inhibition in the other three bacteria.

TABLE 9
Evaluation of the germicidal activity of curcumin water solution
pH 6.5 after 30 seconds and 10 minutes of exposure.

| | | 30 Seconds | | 10 Minutes | |
|---|---|---|---|---|---|
| | Challenge | No. cfu | Percent | No. cfu | Percent |
| Microorganism | spc × 10⁶ | recovered | reduction | recovered | reduction |
| Staphylococcus aureus | 150 × 10⁶ | 910,000 | 39.33 | 1,100,000 | 26.67 |
| ATCC 29740 | | | | | |
| Streptococcus agalactiae | 10 × 10⁶ | 8,300 | 91.7 | 300 | 99.7 |
| ATCC 27956 | | | | | |
| Streptococcus uberis | 40 × 10⁶ | 830,000 | 0 | 540,000 | 0 |
| ATCC 27958 | | | | | |
| Escherichia coli | 110 × 10⁶ | 1,400,000 | 0 | 16,000 | 98.54 |
| ATCC 25922 | | | | | |
| Pseudomonas aeruginosa | 130 × 10⁶ | 100 | 99.99 | 0 | >99.99 |
| ATCC 27853 | | | | | |
| Enterobacter aerogenes | 96 × 10⁶ | 1,700,000 | 0 | 1,300,000 | 0 |
| (A216RF) | | | | | |
| Klebsiella pneumoniae | 37 × 10⁶ | 590,000 | 0 | 500,000 | 0 |
| (A37RR) | | | | | |
| Streptococcus dysgalactiae | 48 × 10⁶ | 270,000 | 43.75 | 250,000 | 47.92 |
| ATCC 27957 | | | | | |

Second Experiment. For the second experiment, the bacterial cultures were as described above. The curcumin sample (Batch# CUR-SFA5-031209) was made as follows. First, 100 mL of a 5% w/v rubusoside water solution was prepared. This solubilizing solution was added to 24 mg of curcumin (Cayman Chemical, Ann Arbor, Mich.), mixed and sonicated for 60 min at 60° C. This solution was then autoclaved at 115° C. and 1 atmosphere pressure for 30 min. The autoclaved solution was held in an incubator at 37° C. for 72 hr, and kept in the dark. The solution was adjusted to pH 7.4 by adding appropriate amount of phosphate buffered saline powder and then filtered through a 0.45 μm Nylon filter and analyzed on HPLC. The curcumin concentration in this solution was 157 μg/ml.

The cultured solution had a pH of 7.4 as a result of PBS adjustment of the solubilized curcumin water solution. In contrast to the curcumin/rubusoside solution with pH of 6.1, this curcumin/rubusoside solution with pH 7.4 retained inhibitory activity against all eight bacteria within 30 seconds and lasting through 10 min ranging from 60% to 94%. The results are shown in Table 10. These results indicate the importance of pH in using the solubilized curcumin solution as a bacteriocide.

TABLE 10
Evaluation of the germicidal activity of curcumin water solution
pH 7.4 after 30 seconds and 10 minutes of exposure.

| | | 30 Seconds | | 10 Minutes | |
|---|---|---|---|---|---|
| | Challenge | No. cfu | Percent | No. cfu | Percent |
| Microorganism | spc × 10⁶ | recovered | reduction | recovered | reduction |
| Staphylococcus aureus | 840 × 10⁶ | 1,900,000 | 77.38 | 1,100,000 | 86.9 |
| ATCC 29740 | | | | | |
| Streptococcus agalactiae | 120 × 10⁶ | 270,000 | 77.5 | 110,000 | 90.83 |
| ATCC 27956 | | | | | |
| Streptococcus uberis | 370 × 10⁶ | 210,000 | 94.32 | 480,000 | 87.03 |
| ATCC 27958 | | | | | |
| Escherichia coli | 970 × 10⁶ | 3,100,000 | 68.04 | 2,600,000 | 73.20 |
| ATCC 25922 | | | | | |
| Pseudomonas aeruginosa | 2,980 × 10⁶ | 2,900,000 | 90.27 | 2,100,000 | 92.95 |
| ATCC 27853 | | | | | |
| Enterobacter aerogenes | 770 × 10⁶ | 1,400,000 | 81.82 | 600,000 | 92.2 |
| (A216RF) | | | | | |
| Klebsiella pneumoniae | 820 × 10⁶ | 800,000 | 90.24 | 1,200,000 | 85.37 |
| (A37RR) | | | | | |
| Streptococcus dysgalactiae | 250 × 10⁶ | <100,000 | >60 | <100,000 | >60 |
| ATCC 27957 | | | | | |

Experiment Three. The minimum inhibitory concentration of the curcumin (from Experiment 2) was determined by a standard broth dilution procedure. Two ml tubes of Mueller Hinton broth were prepared, and nine tubes were used for each organism. Two ml of the stock curcumin mixture (157 μg/ml) was added to the first tube resulting in a 1:2 dilution of the curcumin. A two ml aliquot was removed from tube 1 and added to tube 2 resulting in an additional 1:2 dilution, resulting in a total dilution of 1:4. This pattern was repeated through 8 tubes with the final tube acting as a control tube with no curcumin added. After the dilution step, a standardized concentration of organisms in a 0.01 ml volume (approximately 100,000 cfu) incubation tube were observed for lack of turbidity indicating inhibition of growth. Tubes with no visible growth were sub-cultured to determine if growth was merely inhibited or actual killing of the organisms occurred.

MIC varied with bacteria (Table 11) ranging from 19 μg/ml (against Streptococcus agalactiae) to 78 μg/ml (against Staphylococcus aureus, Streptococcus uberis, and Enterobacter aerogenes).

TABLE 11
Minimum inhibitory concentration (MIC) and minimum bacteriacidal
concentration (MBC) of Curcumin in rubusoside water solutions

| Microorganism | MIC (μg/ml) | MBC (μg/ml) |
|---|---|---|
| Staphylococcus aureus | 78.5 | >78.5 |
| ATCC 29740 | | |
| Streptococcus agalactiae | 19.0 | 39.25 |
| ATCC 27956 | | |
| Streptococcus uberis | 78.5 | >78.5 |
| ATCC 27958 | | |
| Escherichia coli | 39.25 | >78.5 |
| ATCC 25922 | | |
| Pseudomonas aeruginosa | 39.25 | >78.5 |
| ATCC 27853 | | |
| Enterobacter aerogenes | 78.5 | >78.5 |
| (A216RF) | | |
| Klebsiella pneumoniae | 39.25 | 78.5 |
| (A37RR) | | |
| Streptococcus dysgalactiae | 39.25 | 39.25 |
| ATCC 27957 | | |

### Example 19

### Effect of Rubusoside on the Water Solubility of Itraconazole and Celecoxib

A 10% w/v rubusoside (isolated as described above) water solution was prepared. Six milligrams and 2.5 mg of itraconazole were weighed into two separate tubes, respectively. Into these two samples were added 5 mL of the rubusoside water solution and 5 mL distilled and deionized water, respectively. Approximately 6.7 mg and 3.7 mg of celecoxib were weighed into two separate tubes, respectively. Into these two samples were added 5 mL of the rubusoside water solution and 5 mL distilled and deionized water, respectively. The solutions were sonicated at 50° C. for 60 min followed by incubation in a water bath at 80° C. for 30 min, and then incubation at 25° C. for 24 hr. The solutions were analyzed on HPLC after filtering with 0.45 μm filters, using each compound in methanol solutions (itraconazole at 180 μg/mL and celecoxib at 420 μg/mL) as standard solutions for quantification.

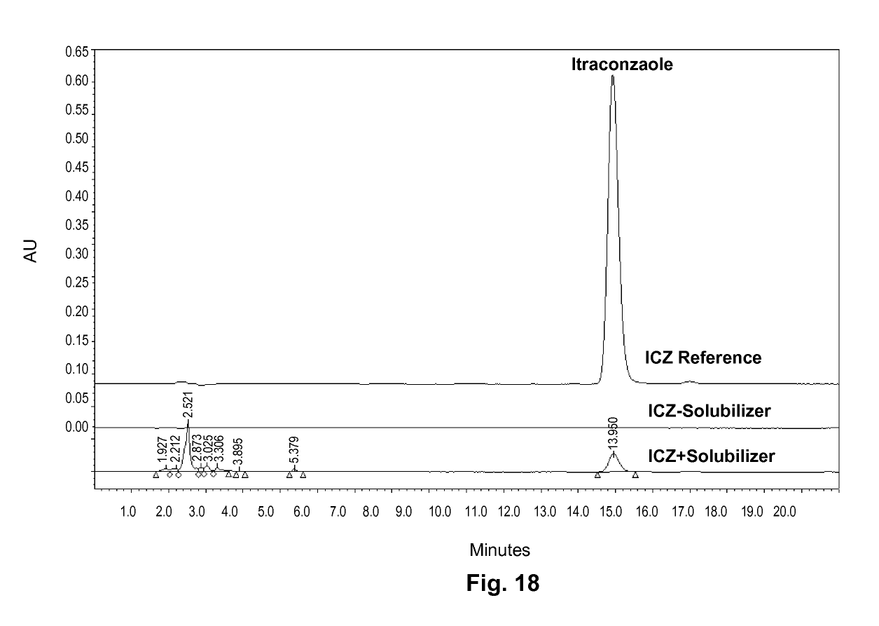

Chromatograms of three itraconazole (ICZ) samples by HPLC-PDA are shown in FIG. 18. A Luna C18 column was used for the HPLC analyses. The mobile phase consisted of acetonitrile (A) and water (B). All the chromatograms were obtained at 260 nm. In FIG. 18, “ICZ+Solubilizer” is the water solution of itraconazole in the presence of 10% rubusoside. “ICZ−Solubilizer” is the water solution of itraconazole without rubusoside. “ICZ Reference” is the methanol solution of itraconazole at 180 μg/ml. In the presence of 10% w/v rubusoside, itraconazole in the aqueous solution (pH=4.09) was 21 μg/mL, whereas itraconazole in the aqueous solution without rubusoside was not detected (FIG. 18).

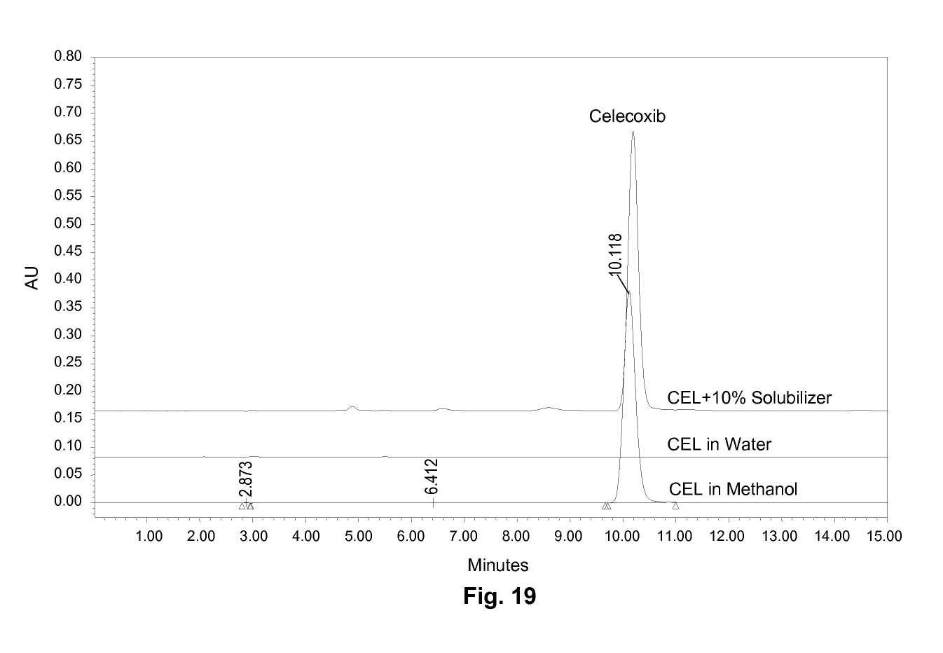

Chromatograms of the three celecoxib (CEL) samples by HPLC-PDA are shown in FIG. 19. A Luna C18 column was used for the HPLC analyses, and the mobile phase consisted of methanol (A) and water (B). All the chromatograms were obtained at 254 nm. In FIG. 19, “CEL+10% solubilizer” is the water sample of celecoxib in the presence of 10% w/v solubilizer (rubusoside); “CEL in water” is the water sample of celecoxib without solubilizer; and “CEL in methanol” is celecoxib methanol solution of 420 μg/mL, used as a standard. In the presence of 10% w/v rubusoside, celecoxib in the aqueous solution was 488 μg/mL, whereas celecoxib in the aqueous solution without rubusoside was not detected (FIG. 21).

The complete disclosures of all references cited in this specification are hereby incorporated by reference, including U.S. provisional patent applications Ser. Nos. 61/044,176 and 61/099,823. In the event of an otherwise irreconcilable conflict, however, the present specification shall control.

1. A method for enhancing the solubility of an organic compound which is insoluble or sparingly soluble in water, said method comprising mixing said compound with water and with a diterpene glycoside in a concentration sufficient to increase the solubility of the compound in water by a factor of 2 or more.

2. The method of claim 1, wherein the diterpene glycoside is selected from the group consisting of rubuososide, stevioside, rebaudioside A, rebaudioside B, rebaudioside C, rebaudioside D, rebaudioside E, rebaudioside F, steviol monoside, dulcoside A, steviol bioside, paniculoside, suavioside A, suavioside B, suavioside C1, suavioside D1, suavioside D2, suavioside E, suavioside F, suavioside G, suavioside H, suavioside I, suavioside J, goshonoside F1, goshonoside F2, goshonoside F3, goshonoside F4, and goshonoside F5.

3. The method of claim 1, wherein the diterpene glycoside is rubusoside.

4. The method of claim 1, wherein the diterpene glycoside is rebaudioside A.

5. The method of claim 1, wherein the diterpene glycoside is stevioside.

6. The method of claim 1, wherein the diterpene glycoside is steviol monoside.

7. The method of claim 1, wherein the compound is selected from the group consisting of diterpenes, quinoline alkaloids, phenylalanine-derived alkaloids, hydrolysable tannins, flavonoids, curcuminoids, phenols, quinones, macrolides, cyclic peptides, sesquiterpene lactones, lignans, flavonolignans, lipids, and azoles.

8. The method of claim 1, wherein the compound is a diterpene selected from the group consisting of paclitaxel, docetaxel, baccatin III, 10-deacetylbaccatin III, cephalomannine, 10-deacetylcephalomannine, retinoids, ginkgolide, and forsakolin.

9. The method of claim 1, wherein the compound is paclitaxel.

10. The method of claim 1, wherein the compound is a quinoline alkaloid selected from the group consisting of camptothecin, 10-hydroxycamptothecin, methoxycamptothecin, 9-nitrocamptothecin, quinine, quinidine, cinchonidine, and cinchonine.

11. The method of claim 1, wherein the compound is camptothecin.

12. The method of claim 1, wherein the compound is a phenylalanine-derived alkaloid selected from the group consisting of capsaicin and dihydrocapsaicin.

13. The method of claim 1, wherein the compound is capsaicin.

14. The method of claim 1, wherein the compound is a hydrolysable tannin selected from the group consisting of gallic acid and ellagic acid.

15. The method of claim 1, wherein the compound is gallic acid.

16. The method of claim 1, wherein the compound is a flavonoid selected from the group consisting of flavonones, flavones, dihydroflavonols, flavonols, flavandiols, leucoanthocyanidins, flavonol glycosides, flavonone glycosides, isoflavonoids, and neoflavonoids.

17. The method of claim 1, wherein the compound is a flavonoid selected from the group consisting of naringenin, eriodictyol, apigenin, luteolin, dihydrokaempferol, dihydroquercetin, kaempferol, quercetin, leucopelargonidin, leucocyanidin, rutin, hesperidin, neohesperidin naringin, daidzein, genistein, coumestrol, rotenone, and pisatin.

18. The method of claim 1, wherein the compound is rutin.

19. The method of claim 1, wherein the compound is a curcuminoid selected from the group consisting of curcumin, desmethoxycurcumin, and bis-desmethoxycurcumin.

20. The method of claim 1, wherein the compound is curcumin.

21. The method of claim 1, wherein the compound is a phenol selected from the group consisting of tocopherol, propofol, and gingerol.

22. The method of claim 1, wherein the compound is alpha-tocopherol.

23. The method of claim 1, wherein the compound is propofol.

24. The method of claim 1, wherein the compound is gingerol.

25. The method of claim 1, wherein the compound is a quinone selected from the group consisting of ubiquinones, plastoquinones, anthraquinones, phenanthraquinones, and di-anthraquinones.

26. The method of claim 1, wherein the compound is a quinone selected from the group consisting of coenzyme Q, coenzyme Q10, rhein, emodin, alizarin, lucidin, cryptotanshinone, tanshinone I, tanshinone IIA, dihydrotanshinone, sennoside A, and sennoside B.

27. The method of claim 1, wherein the compound is coenzyme Q10.

28. The method of claim 1, wherein the compound is tanshinone IIA.

29. The method of claim 1, wherein the compound is a macrolide selected from the group consisting of erythromycin, oleandomycin, spiramycin I, spiramycin II, spiramycin III, tylosin, avermectins, amphotericin B, nystatin, tacrolimus, and rapamycin.

30. The method of claim 1, wherein the compound is erythromycin.

31. The method of claim 1, wherein the compound is amphotericin B.

32. The method of claim 1, wherein the compound is nystatin.

33. The method of claim 1, wherein the compound is rapamycin.

34. The method of claim 1, wherein the compound is a cyclic peptide selected from the group consisting of cyclosporine, polymyxin, tyrothricin, gramicidins, capreomycin, vancomycin, cephalosporin, and cephamycin.

35. The method of claim 1, wherein the compound is cyclosporin A.

36. The method of claim 1, wherein the compound is a sesquiterpene lactone selected from the group consisting of artemisinin, dihydroartemisinin, and bilobalide.

37. The method of claim 1, wherein the compound is artemisinin.

38. The method of claim 1, wherein the compound is a lignan selected from the group consisting of podophyllotoxin, 4′-demethylpodophyllotoxin, beta-peltatin, alpha-peltatin, desoxypodophyllotoxin, podophyllotoxone, matairesinol, yatein, and pinoresinol.

39. The method of claim 1, wherein the compound is podophyllotoxin.

40. The method of claim 1, wherein the compound is a flavonolignan selected from the group consisting of silybin, isosilybin, and silychristin.

41. The method of claim 1, wherein the compound is silybin.

42. The method of claim 1, wherein the compound is a component of fish oil.

43. The method of claim 42, wherein the component of fish oil is a fatty acid.

44. The method of claim 1, wherein the compound is an azole selected from the group consisting of itraconazole, fluconazole, isavuconazole, voriconazole, pramiconazole, posaconazole, ravuconazole, fluconazole, fosfluconazole, epoxiconazole, triadimenol, propiconazole, metconazole, cyproconazole, tebuconazole, flusilazole, paclobutrazol, and celecoxib.

45. The method of claim 1, wherein the compound is itraconazole.

46. The method of claim 1, wherein the compound is celecoxib.

47. A composition comprising an aqueous solution of an organic compound having low solubility in water, and a diterpene glycoside; wherein the concentration of said diterpene glycoside is sufficient to increase the solubility of said compound in water by a factor of 2 or more above what the solubility of said compound would be in an otherwise identical composition lacking said diterpene glycoside.

48. The composition of claim 47, wherein said diterpene glycoside is selected from the group consisting of rubuososide, stevioside, rebaudioside A, rebaudioside B, rebaudioside C, rebaudioside D, rebaudioside E, rebaudioside F, steviol monoside, dulcoside A, steviol bioside, paniculoside, suavioside A, suavioside B, suavioside C1, suavioside D1, suavioside D2, suavioside E, suavioside F, suavioside G, suavioside H, suavioside I, suavioside J, goshonoside F1, goshonoside F2, goshonoside F3, goshonoside F4, and goshonoside F5.

49. The composition of claim 47, wherein the diterpene glycoside is rubusoside.

50. The composition of claim 47, wherein the diterpene glycoside is rebaudioside A.

51. The composition of claim 47, wherein the diterpene glycoside is stevioside.

52. The composition of claim 47, wherein the diterpene glycoside is steviol monoside.

53. The composition of claim 47, wherein the compound is selected from the group consisting of diterpenes, quinoline alkaloids, phenylalanine-derived alkaloids, hydrolysable tannins, flavonoids, curcuminoids, phenols, quinones, macrolides, cyclic peptides, sesquiterpene lactones, lignans, flavonolignans, lipids, and azoles.

54. The composition of claim 47, wherein the compound is a diterpene selected from the group consisting of paclitaxel, docetaxel, baccatin III, 10-deacetylbaccatin III, cephalomannine, 10-deacetylcephalomannine, ginkgolide, and forsakolin.

55. The composition of claim 47, wherein the compound is paclitaxel.

56. The composition of claim 47, wherein the compound is a quinoline alkaloid selected from the group consisting of camptothecin, 10-hydroxycamptothecin, methoxycamptothecin, 9-nitrocamptothecin, quinine, quinidine, cinchonidine, and cinchonine.

57. The composition of claim 47, wherein the compound is camptothecin.

58. The composition of claim 47, wherein the compound is a phenylalanine-derived alkaloid selected from the group consisting of capsaicin and dihydrocapsaicin.

59. The composition of claim 47, wherein the compound is capsaicin.

60. The composition of claim 47, wherein the compound is a hydrolysable tannin selected from the group consisting of gallic acid and ellagic acid.

61. The composition of claim 47, wherein the compound is gallic acid.

62. The composition of claim 47, wherein the compound is a flavonoid selected from the group consisting of flavonones, flavones, dihydroflavonols, flavonols, flavandiols, leucoanthocyanidins, flavonol glycosides, flavonone glycosides, isoflavonoids, and neoflavonoids.

63. The composition of claim 47, wherein the compound is a flavonoid selected from the group consisting of naringenin, eriodictyol, apigenin, luteolin, dihydrokaempferol, dihydroquercetin, kaempferol, quercetin, leucopelargonidin, leucocyanidin, rutin, hesperidin, neohesperidin naringin, daidzein, genistein, coumestrol, rotenone, and pisatin.

64. The composition of claim 47, wherein the compound is rutin.

65. The composition of claim 47, wherein the compound is a curcuminoid selected from the group consisting of curcumin, desmethoxycurcumin, and bis-desmethoxycurcumin.

66. The composition of claim 47, wherein the compound is curcumin.

67. The composition of claim 47, wherein the compound is a phenol selected from the group consisting of tocopherol, propofol, and gingerol.

68. The composition of claim 47, wherein the compound is alpha-tocopherol.

69. The composition of claim 47, wherein the compound is propofol.

70. The composition of claim 47, wherein the compound is gingerol.

71. The composition of claim 47, wherein the compound is a quinone selected from the group consisting of ubiquinones, plastoquinones, anthraquinones, phenanthraquinones, and di-anthraquinones.

72. The composition of claim 47, wherein the compound is a quinone selected from the group consisting of coenzyme Q, coenzyme Q10, rhein, emodin, alizarin, lucidin, cryptotanshinone, tanshinone I, tanshinone IIA, dihydrotanshinone, sennoside A, and sennoside B.

73. The composition of claim 47, wherein the compound is coenzyme Q10.

74. The composition of claim 47, wherein the compound is tanshinone IIA.

75. The composition of claim 47, wherein the compound is a macrolide selected from the group consisting of erythromycin, oleandomycin, spiramycin I, spiramycin II, spiramycin III, tylosin, avermectins, amphotericin B, nystatin, tacrolimus, and rapamycin.

76. The composition of claim 47, wherein the compound is erythromycin.

77. The composition of claim 47, wherein the compound is amphotericin B.

78. The composition of claim 47, wherein the compound is nystatin.

79. The composition of claim 47, wherein the compound is rapamycin.

80. The composition of claim 47, wherein the compound is a cyclic peptide selected from the group consisting of cyclosporine, polymyxin, tyrothricin, gramicidins, capreomycin, vancomycin, cephalosporin, and cephamycin.

81. The composition of claim 47, wherein the compound is cyclosporin A.

82. The composition of claim 47, wherein the compound is a sesquiterpene lactone selected from the group consisting of artemisinin, dihydroartemisinin, and bilobalide.

83. The composition of claim 47, wherein the compound is artemisinin.

84. The composition of claim 47, wherein the compound is a lignan selected from the group consisting of podophyllotoxin, 4′-demethylpodophyllotoxin, beta-peltatin, alpha-peltatin, desoxypodophyllotoxin, podophyllotoxone, matairesinol, yatein, and pinoresinol.

85. The composition of claim 47, wherein the compound is podophyllotoxin.

86. The composition of claim 47, wherein the compound is a flavonolignan selected from the group consisting of silybin, isosilybin, and silychristin.

87. The composition of claim 47, wherein the compound is silybin.

88. The composition of claim 47, wherein the compound is a component of fish oil.

89. The composition of claim 88, wherein the component of fish oil is a fatty acid.

90. The composition of claim 47, wherein the compound is an azole selected from the group consisting of itraconazole, fluconazole, isavuconazole, voriconazole, pramiconazole, posaconazole, ravuconazole, fluconazole, fosfluconazole, epoxiconazole, triadimenol, propiconazole, metconazole, cyproconazole, tebuconazole, flusilazole, paclobutrazol and celecoxib.

91. The composition of claim 47, wherein the compound is itraconazole.

92. The composition of claim 47, wherein the compound is celecoxib.

93. The composition of claim 47, wherein said compound is gallic acid and, wherein said diterpene glycoside is rubusoside.

94. The composition of claim 47, wherein said compound is curcumin and, wherein said diterpene glycoside is rubusoside.

95. The composition of claim 47, wherein said compound is curcumin and, wherein said diterpene glycoside is rubusoside and rebaudioside A.

96. The composition of claim 47, wherein said compound is camptothecin and, wherein said diterpene glycoside is rubusoside.

97. The composition of claim 47, wherein said compound is capsaicin and, wherein said diterpene glycoside is rubusoside.

98. The composition of claim 47, wherein said compound is paclitaxel and, wherein said diterpene glycoside is rubusoside.

99. The composition of claim 47, wherein said compound is rutin and, wherein said diterpene glycoside is rubusoside.

100. The composition of claim 47, wherein said compound is tanshinone IIA and, wherein said diterpene glycoside is rubusoside.

101. The composition of claim 47, wherein said compound is amphotericin B and, wherein said diterpene glycoside is rubusoside.

102. The composition of claim 47, wherein said compound is cyclosporin and, wherein said diterpene glycoside is rubusoside.

103. The composition of claim 47, wherein said compound is nystatin and, wherein said diterpene glycoside is rubusoside.

104. The composition of claim 47, wherein said compound is erythromycin and, wherein said diterpene glycoside is rubusoside.

105. The composition of claim 47, wherein said compound is coenzyme Q10 and, wherein said diterpene glycoside is rubusoside.

106. The composition of claim 47, wherein said compound is propofol and, wherein said diterpene glycoside is rubusoside.

107. The composition of claim 47, wherein said compound is artemisinin and, wherein said diterpene glycoside is rubusoside.

108. The composition of claim 47, wherein said compound is podophyllotoxin and, wherein said diterpene glycoside is rubusoside.

109. The composition of claim 47, wherein said compound is alpha-tocopherol and, wherein said diterpene glycoside is rubusoside.

110. The composition of claim 47, wherein said compound is silybin and, wherein said diterpene glycoside is rubusoside.

111. The composition of claim 47, wherein said compound is rapamycin and, wherein said diterpene glycoside is rubusoside.

112. The composition of claim 47, wherein said compound is gingerol and, wherein said diterpene glycoside is rubusoside.

113. The composition of claim 47, wherein said compound is itraconazole and, wherein said diterpene glycoside is rubusoside.

114. The composition of claim 47, wherein said compound is celecoxib and, wherein said diterpene glycoside is rubusoside.

115. The composition of claim 47, additionally comprising one or more pharmaceutical agents selected from the group consisting of complexing agents, cosolvents, surfactants, emulsifiers, liposomes and nanoparticles.
